(19) United States
(12) Patent Application Publication
(10) Pub. No.: US 2021/0233658 A1
(43) Pub. Date: Jul. 29, 2021

(54) Identifying Relevant Medical Data for Facilitating Accurate Medical Diagnosis

(71) Applicant: Babylon Partners Limited, London (GB)

(72) Inventors: Camille Van Assel, London (GB); Domenico Corapi, London (GB); Maurizio Morriello, London (GB); Miklos Kepes, London (GB); Joshua Samuel Nathan Levy-Kramer, London (GB); Nils Yannick Hammerla, London (GB)

(21) Appl. No.: 16/750,885

(22) Filed: Jan. 23, 2020

(51) Int. Cl. G16H 50/20; G16H 10/60; G16H 50/30; G16H 70/60; G16H 15/00

(52) U.S. Cl. CPC G16H 50/20; G16H 10/60; G16H 15/00; G16H 70/60; G16H 50/30

(57) ABSTRACT

A computer-implemented method for medical diagnosis, comprising:
receiving a user input from a user, the user input comprising an input symptom;
determining a measure of relevance of a plurality of items of medical data to the user input, wherein the plurality of items of medical data are items of medical data for which information associated with the user is stored;
determining whether to include the stored information corresponding to an item of medical data in a first set of information, based on the measure of relevance for the item of medical data;
providing the user input and the first set of information as an input to a model, the model being configured to output a probability of the user having a disease; and
outputting a diagnosis based on the probability of the user having a disease.

### FIELD

Embodiments described herein relate to methods and systems for medical diagnosis, and methods for training medical diagnosis systems. In particular, such methods and systems for medical diagnosis may determine a probability of a disease from information input by a user and from information retrieved from the user's clinical history.

### BACKGROUND

Medical diagnosis systems may use knowledge of symptoms experienced by a patient, combined with information regarding risk factors for example, to identify medical conditions (diseases). This may allow offering of possible treatments to the patient.

In many cases, medical diagnosis is based on making a decision by considering the causal and probabilistic relationship between items of medical data such as risk-factors, diseases and symptoms. Medical models may be used to describe the interplay between items of medical data. For example, a model that elegantly captures such causal relationships is based on the framework of probabilistic graphical models (PGM). Key to decision-making in such a system is the process of performing probabilistic inference on the PGM.

Such systems determine the likelihood of a set of diseases, based on available evidence. The available evidence is provided by a user. However, the evidence provided may be incomplete. With incomplete evidence, the likelihood of diseases may be difficult to predict and the accuracy of the diagnosis may be poor.

### BRIEF DESCRIPTION OF FIGURES

The patent or application file contains at least one drawing executed in color. Copies of this patent or patent application publication with color drawing(s) will be provided by the Office upon request and payment of the necessary fee.

Systems and methods in accordance with non-limiting embodiments will now be described with reference to the accompanying figures in which:

FIG. 1(a) is a schematic illustration of an exemplary medical diagnosis system;

FIG. 1(b) is a visualization of part of a knowledge graph for the descendant concepts of the concept “acute asthma”;

FIG. 1(c) is a visualisation of part of a knowledge graph for the descendant concepts of the concept “diabetes type 1”;

FIG. 2(a) is a schematic illustration of a medical diagnosis system in accordance with an embodiment;

FIG. 2(b) is a schematic illustration of a module for determining a measure of relevance of medical information, which may be included in a medical diagnosis system in accordance with an embodiment;

FIG. 2(c) shows a flow chart illustrating a method for determining a measure of relevance of medical information, which may be used in a method of medical diagnosis in accordance with an embodiment;

FIG. 2(d) illustrates an example of how the relevant clinical history may be presented to a user on a user device;

FIG. 3(a) is a depiction of a probabilistic graphical model (PGM) used in a medical diagnosis system in accordance with an embodiment;

FIG. 3(b) illustrates an importance sampling method used in a medical diagnosis method in accordance with an embodiment;

FIG. 3(c) illustrates an example training process for a universal marginalizer (UM) used in a medical diagnosis system in accordance with an embodiment;

FIG. 4 is a schematic illustration of a medical diagnosis system in accordance with an embodiment;

FIG. 5 illustrates a method for medical diagnosis in accordance with an embodiment;

FIG. 6(a) is a schematic illustration of a module for determining the validity and a measure of the relevance of medical information, which may be included in a medical diagnosis system in accordance with an embodiment;

FIG. 6(b) shows a flow chart illustrating a method for determining the validity of medical information which may be used in a method of medical diagnosis in accordance with an embodiment;

FIG. 6(c) shows a flow chart of a method for determining the validity of medical information which may be used in a method of medical diagnosis in accordance with an embodiment;

FIG. 7(a) shows a flow chart of a method of training a medical diagnosis system in accordance with an embodiment, the method comprising generating vectors corresponding to medical concepts;

FIG. 7(b) shows a schematic illustration of an example model which may be used in the method of FIG. 7(a);

FIG. 7(c) shows an overview of the content of an example Electronic Health Record database, which may be used to generate a training dataset in the method of FIG. 7(a);

FIG. 8(a) shows a flow chart of a method of training a medical diagnosis system in accordance with an embodiment, the method comprising generating vectors corresponding to medical concepts;

FIG. 8(b) shows a visualisation of the process of extracting similar nodes used in the method of FIG. 8(a);

FIG. 8(c) shows an example of combined similarity scores for part of a knowledge graph;

FIG. 9(a) shows an example of how relevance might be indicated in a clinical portal displayed on a device to a user;

FIG. 9(b) illustrates an example of how relevance might be indicated in a clinical portal displayed on a device;

FIG. 9(c) is a schematic illustration of an example system for determining a measure of relevance, where the system is a clinical portal system;

FIG. 10(a) shows results of testing to determine a precision measure of the determination of a measure of relevance;

FIG. 10(b) shows further results of testing to determine a precision measure of the determination of a measure of relevance;

FIG. 10(c) shows results of testing to determine a precision measure of a word embeddings based determination of a measure of relevance;

FIG. 11 is a schematic illustration of a computing system which provides means capable of putting a method for medical diagnosis in accordance with an embodiment into effect.

### DETAILED DESCRIPTION OF FIGURES

According to a first aspect, there is provided a computer-implemented method for medical diagnosis, comprising:
receiving a user input from a user, the user input comprising an input symptom;
determining a measure of relevance of a plurality of items of medical data to the user input, wherein the plurality of items of medical data are items of medical data for which information associated with the user is stored;
determining whether to include the stored information corresponding to an item of medical data in a first set of information, based on the measure of relevance for the item of medical data;
providing the at least one input symptom and the first set of information as an input to a model, the model being configured to output a probability of the user having a disease; and
outputting a diagnosis based on the probability of the user having a disease.

According to a second aspect, there is provided a medical diagnosis system comprising:
a user interface configured to receive a user input from a user, the user input comprising at least one input symptom;
one or more processors configured to:
   determine a measure of relevance of a plurality of items of medical data to the user input, wherein the plurality of items of medical data are items of medical data for which information associated with the user is stored;
   determine whether to include the information corresponding to an item of medical data in a first set of information, based on the measure of relevance for the item of medical data;
   provide the at least one input symptom and the first set of information as an input to a model, the model being configured to output a probability of the user having a disease; and
a display device, configured to display a diagnosis based on the probability of the user having a disease.

The disclosed system addresses a technical problem tied to computer technology and arising in the realm of computer networks, namely the technical problem of efficiency of data transmission. In a medical diagnosis system, information from a stored user medical history may be used to improve the accuracy of diagnosis. However, information from the medical history may be incorrect, or may no longer be correct. The medical diagnosis system therefore transmits the medical history information to the user for confirmation. However, the medical history may comprise a large amount of information, resulting in a large amount of data being transmitted to the user. The disclosed system solves this technical problem by filtering the medical history information based on relevance, to obtain a sub-set of the information. The sub-set of information comprises information determined to be relevant to the presenting symptom. By filtering the medical history information based on relevance, a sub-set of the medical history data is transmitted, whilst an accurate diagnosis may still be obtained.

In an embodiment, determining the measure of the relevance of an item of medical data to the user input comprises:
obtaining a first vector representation corresponding to the input symptom;
obtaining a second vector representation corresponding to the item of medical data;
determining a similarity measure between the first vector representation and the second vector representation. The similarity measure may be the cosine similarity.

The vector representations corresponding to items of medical data are generated during a training stage, and then stored.

The user input may comprise two or more input symptoms, or other items of medical data. In an embodiment, determining the measure of the relevance of an item of medical data to the user input comprises:
obtaining a first vector representation corresponding to each of the input items;
obtaining a second vector representation corresponding to the item of medical data;
determining a similarity measure between each first vector representation and the second vector representation; and
determining an average similarity measure.

The input is received from a user device, and the method further comprises sending the first set of information to the user device and receiving confirmation information corresponding to the first set of information from the user device. The confirmed information is then input into the model.

Determining whether to include the information corresponding to an item of medical data in a first set of information based on the measure of relevance for the item of medical data may comprise determining whether the measure of relevance meets a pre-determined threshold. Alternatively, determining whether to include the information corresponding to an item of medical data in a first set of information based on the measure of relevance for the item of medical data may comprise determining whether the measure of relevance is within a pre-determined number of the most relevant.

An item of medical data comprises a symptom, risk factor, disease, physiological data, recommendation or behaviour.

In an embodiment, the model comprises a probabilistic graphical model comprising probability distributions and relationships between symptoms and diseases, and an inference engine configured to perform Bayesian inference on said probabilistic graphical model, and wherein determining the probability that the user has a disease comprises performing approximate inference on the probabilistic graphical model to obtain a prediction of the probability that the user has a disease.

In an embodiment, the method further comprises:
obtaining a set of items of medical data to be used in the probabilistic graphical model;
obtaining stored information associated with the user relating to the items of medical data to be used in the model;
determining the measure of the relevance for the items of medical data.

In an embodiment, inference is performed using a discriminative model, wherein the discriminative model has been pre-trained to approximate the probabilistic graphical model, the discriminative model being trained using samples generated from said probabilistic graphical model, wherein some of the data of the samples has been masked to allow the discriminative model to produce data which is robust to the user providing incomplete information about their symptoms,

and wherein determining the probability that the user has a disease comprises deriving estimates of the probabilities that the user has that disease from the discriminative model, inputting these estimates to the inference engine and performing approximate inference on the probabilistic graphical model to obtain a prediction of the probability that the user has that disease.

In an embodiment, the method further comprises:
determining the validity of the stored information;
wherein determining the measure of the relevance of the plurality of items of medical data comprises determining the measure of relevance for the items of medical data for which the stored information is valid.

The validity may be determined from information indicating which items of medical data are permanently valid.

According to a third aspect, there is provided a computer implemented method of training a medical diagnosis system, comprising:
obtaining a dataset comprising a plurality of items of medical data associated with each of a plurality of patients;
learning a vector representation corresponding to items of medical data in the dataset;
storing the vector representation associated with the item of medical data.

The vector representations may be learned by training a model to reconstruct the context of concepts from the dataset. For example, a word2vec or fasttext based model architecture may be used.

In an embodiment, the method further comprises:
obtaining an ontology comprising items of medical data and information describing the relationships between the items of medical data;
for a target item of medical data, determining one or more items of medical data in the ontology which are similar to the target item and for which there is an associated stored vector representation;
determining a vector representation for the target item of medical data from the vector representations of the one or more similar items of medical data.

An item of medical data may be determined to be similar to a target item using an information content based similarity measure.

In an embodiment, the method further comprises training a discriminative model to approximate the output of a probabilistic graphical model, comprising:
receiving by the discriminative model samples from said probabilistic graphical model; and
training the discriminative model using said samples,
wherein some of the data of the samples has been masked to allow the deterministic model to produce data which is robust to the user failing to input at least one symptom. In an further embodiment, the masking is based on a uniform distribution.

According to a fourth aspect, there is provided a computer implemented method for determining a measure of relevance, the method comprising:
determining a measure of relevance of a plurality of items of medical data to a target item of medical data, wherein the plurality of items of medical data are items of medical data for which information associated with the user is stored, wherein determining the measure of the relevance of an item of medical data to the target item of medical data comprises:
obtaining a first vector representation corresponding to the target item of medical data;
obtaining a second vector representation corresponding to the item of medical data;
determining a similarity measure between the first vector representation and the second vector representation.

According to a fifth aspect, there is provided a system for determining a measure of relevance, the system comprising a processor, configured to:
determine a measure of relevance of a plurality of items of medical data to a target item of medical data, wherein the plurality of items of medical data are items of medical data for which information associated with the user is stored; wherein determining the measure of the relevance of an item of medical data to the target item of medical data comprises:
obtaining a first vector representation corresponding to the target item of medical data;
obtaining a second vector representation corresponding to the item of medical data;
determining a similarity measure between the first vector representation and the second vector representation.

According to a sixth aspect, there is provided a computer implemented method of training a system for determining a measure of relevance, comprising:
obtaining a dataset comprising a plurality of items of medical data associated with each of a plurality of patients;
learning a vector representation corresponding to items of medical data in the dataset;
storing the vector representation associated with the item of medical data.

According to a seventh aspect, there is provided a carrier medium comprising computer readable code configured to cause a computer to perform the above described methods.

The methods are computer-implemented methods. Since some methods in accordance with embodiments can be implemented by software, some embodiments encompass computer code provided to a general purpose computer on any suitable carrier medium. The carrier medium can comprise any storage medium such as a CD ROM, a magnetic device or a programmable memory device, or any transient medium such as any signal e.g. an electrical, optical or microwave signal. The carrier medium may comprise a non-transitory computer readable storage medium.

The above described systems and methods may determine the likelihood of a set of diseases, based on available evidence, and provide a diagnosis. The available evidence is generated from information provided by a user combined with information relating to the user that is collected from previous interactions (e.g. past diagnoses) and stored in the user's clinical history. Further information may also be requested from the user to provide a more accurate diagnosis. Several requests may be made. Evidence may comprise a list of items of medical data which are present. The items of medical data may be risk-factors, diseases, symptoms, physiological data, recommendations or behaviours. Evidence may further comprise any other information that can be used to provide a diagnosis.

As users interact with the diagnosis system and/or with clinicians for example, hundreds of data points may be generated in the clinical history. For example, a user interacting with the system in order to generate a diagnosis may generate on average 70 data points per patient. A health check interaction may generate around 128 data points, and 8 data points on average may be extracted from clinical notes (such data points may be extracted using NLP techniques on written notes taken by clinicians during consultation for example). In general, the data points correspond to the presence or absence of an item of medical data (e.g. headache). Other data points, such as height or weight entries, may be included however. This information may be used by the diagnosis system to inform the diagnosis.

Information from the medical history may be incorrect, or may no longer be correct. The medical diagnosis system therefore transmits the medical history information to the user for confirmation. However, the medical history may comprise a large amount of information, resulting in a large amount of data being transmitted to the user. By filtering the medical history information based on relevance, to obtain a sub-set of information, comprising information determined to be relevant to the presenting symptom(s), a small amount of data is sent to the user device. Improved efficiency of data transmission to and from the user device is therefore provided.

Furthermore, providing a large set of input data to the diagnosis model, some of which is not relevant to the presenting symptom(s), may result in reduced accuracy of diagnosis in some cases. By filtering the information based on a measure of relevance, an accurate diagnosis may be obtained. A step of pre-processing the clinical history data based on a measure of relevance is performed, to provide an accurate diagnostic. When a patient inputs a presenting complaint (e.g. “I have chest pain”), the pre-processing step returns data from the clinical history that is determined to be relevant to this presenting complaint.

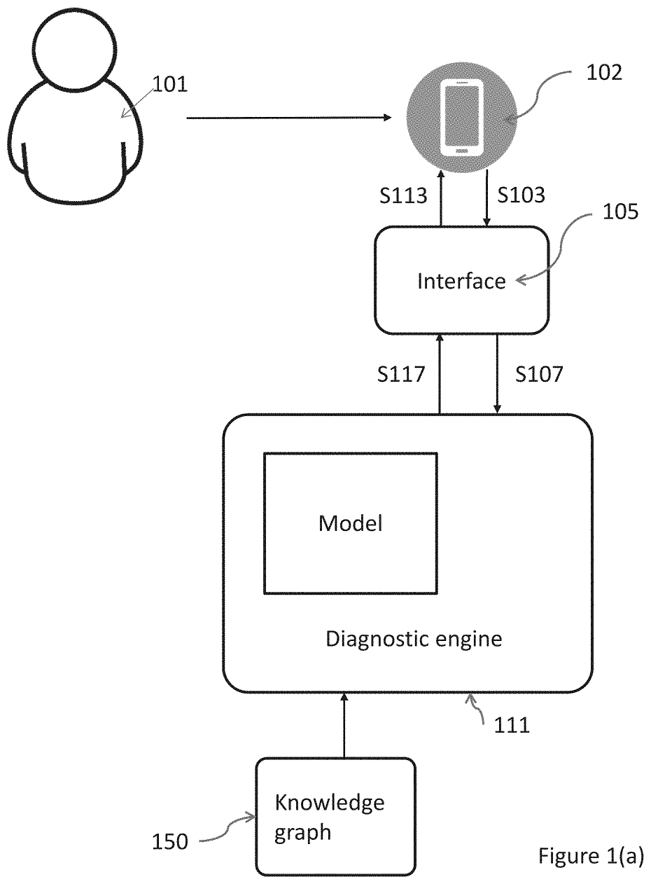

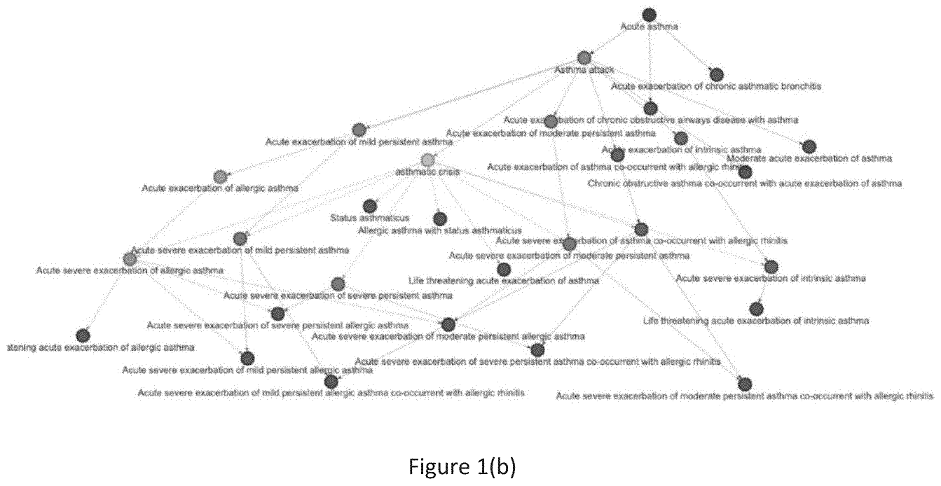

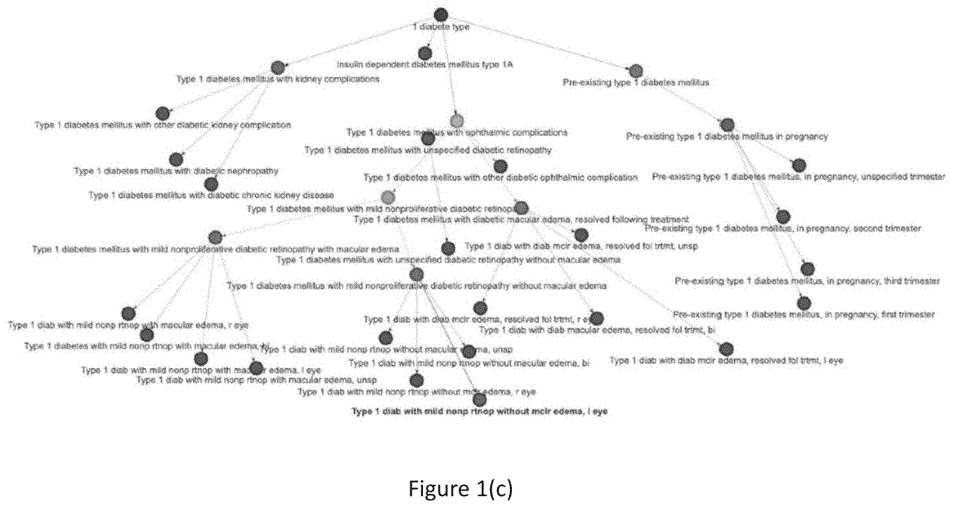

FIG. 1 illustrates an exemplary medical diagnosis system. A patient 101 communicates with the system via a mobile phone device 102. However, any device that is capable of communicating information over a computer network, for example, a laptop, tablet computer, information point, fixed computer etc., could be used. The device 102 may also be referred to as a user terminal. The user terminal is capable of conveying information to the patient in any suitable form. Such forms may include images and text by means of a display, or sounds and speech by means of a loudspeaker. The user terminal may also receive inputs from the user in different forms, for example by the user entering text, selecting from text options displayed on the terminal, or speaking into the device for example.

The mobile phone 102 communicates with interface 105, and transmits the text information corresponding to the user input to the interface 105 in S103. Interface 105 has two primary functions, the first function is to receive the words input by the user. In step S107, this information is passed on to the diagnosis engine 111. The second function is to take the output of the diagnosis engine 111 and to send this back to the user's mobile phone 102 in steps S117 and S113.

The patient 101 inputs their symptom(s) in step via interface 105. The patient may also input other items of medical data which are present, such as their risk factors (for example, whether they are a smoker etc) and/or known diseases (for example they have diabetes or asthma). The interface may be adapted to ask the patient 101 specific questions. Alternately, the patient may simply enter free text.

The diagnostic engine 111 receives the input text information from the interface 105 in step S107. The diagnostic engine then calls an NLP module (not shown) that applies NLP techniques to the input text information to extract concepts. This NLP module converts the natural text into concepts. Concepts will be described in detail below.

For example, in this step, following receipt of an input phrase, the diagnostic engine 111 calls an NLP dependency parser which extracts one or more symptoms which are present in the phrase, and outputs the concept(s) corresponding to the symptom(s). Various known methods of parsing the input text can be used. In this example, the output of the NLP module therefore comprises a set of concepts (in this case representing symptoms) that are present in the input phrase. The sub-set of these concepts corresponding to the items of medical data used in the model is then extracted.

The evidence input into the model may comprise the presence of items of medical data, such as symptoms, diseases, risk factors, physiological data, recommendations and/or behaviours, that are identified as present by the patient in the input. It may further comprise the absence of items of medical data, such as symptoms, diseases, risk factors, physiological data, recommendations and/or behaviours, identified as absent by the patient in the input. In the simple example which is described herein however, the user input evidence comprises a set of one or more symptoms which are present. Items of medical data where the patient has not provided information are assumed to be unknown at this stage.

The evidence is passed in step S107 to the diagnosis engine 111. The diagnosis engine 111 is configured to compute probabilities that diseases are present based on evidence (e.g. the presence of one or more symptoms, diseases and/or risk factors) provided, and derive a diagnosis from these probabilities. The diagnosis engine 111 comprises a model of medicine, which encompasses human knowledge of medicine, and an inference engine, which quantifies the likelihood of a disease being present, in view of the reported evidence (e.g. a list of symptoms, diseases and/or risk factors which are identified as present) and the model of medicine for example. The ‘model of medicine’ may be encoded in several ways, for example it may be a PGM.

The diagnostic engine 111 may then transmit back information in step S117 concerning the “likelihood” of a disease, and generate a diagnosis, given the evidence supplied by the patient 101. The interface 105 can supply this information back to the mobile phone 102 of the patient in step S113. The information may alternatively be outputted to a different device, for example a computer operated by a doctor. The information may then be displayed on a display device for example. For example, the mobile phone 102 of the patient may be configured to display a diagnosis based on the probability of the user having a disease.

The diagnostic engine 111 may be connected to a knowledge graph 150, also referred to as a knowledge base, the knowledge graph being a large structured medical knowledge base, or medical ontology, linking together medical diseases, symptoms, risk factors etc. The knowledge graph can be thought of as a repository of human knowledge on modern medicine encoded in a manner that can be understood by machines. The knowledge graph may keep track of the meaning behind medical terminology across different medical systems and different languages. The diagnostic engine 111 uses the medical knowledge encoded in the knowledge graph and calls the NLP module described above to turn the words input by the user into a form that can be understood by the model, i.e. concepts. Each item of medical data such as a symptom, disease or risk factor for example, corresponds to a concept in the knowledge base 150, as will be described in more detail below.

A visualization of part of the knowledge graph for the descendants of acute asthma is shown in FIG. 1(b). A visualisation of part of the knowledge graph for the descendants of Diabetes Type 1 is shown in FIG. 1(c).

The knowledge graph 150 comprises a set of simple concepts (such as “headache”), encoded using medical information. Each simple concept comprises a label (e.g. “headache”) and an identifier (e.g. an IRI, discussed below). The knowledge graph may be understood to comprise simple concepts, and the relationships between them. Complex concepts may be constructed from two or more simple concepts from the knowledge graph, but are not themselves stored in the knowledge graph. Complex concepts such as “severe headache” may be constructed from a simple concept and one or more modifiers for example.

The relationships may be links between the simple concepts. For example, a simple concept may be “pain”. This simple concept may be a “parent” concept for several “child” simple concepts such as “headache”, etc. These “child” concepts may also be “parent” concepts for further “child” concepts such as “frontal headache”. The child concept “frontal headache” is thus a simple concept that exists in the knowledge graph, and comprises a label and an identifier:

{

“label”: “Frontal headache”,
“iri”: “https://bbl.health/hFEPy0dO2k”

}

Frontal headache in this example is a simple concept which exists in the knowledge base itself. It may also be represented as a complex concept in an alternative formulation however.

“Severe headache” in this example is a complex concept which does not exist in the knowledge graph. The complex concept “severe headache” may be represented as follows:

{

“baseConcept”: {

“label”: “Headache”,
“iri”: “https://bbl.health/eD42RdeKVT”

},
“modifiers”: [

{

“type”: {

“label”: “Severity”,
“iri”: “https://bbl.health/PvSutVtoiC”

},
“value”: {

“label”: “Severe”,
“iri”: “https://bbl.health/m7MbriuuZ8”

}

}

]

}

The complex concept “severe headache” can be created by putting three simple concepts, i.e. three identifiers, from the knowledge base together. The identifiers may identify simple concepts that exist in the knowledge base. In the example of “severe headache” above, the modifier “Severity” is also a simple concept, having a “value” of “severe” in this case. “Severe” is also a simple concept.

Concepts may also include unary concepts, e.g. Not Headache. In this example, “Not” is a special operator. The operator “Not” does not exist in the knowledge base, but it is part of the algebra that is used to define the semantics of the language.

Each simple concept comprises a corresponding identifier that uniquely identifies it. The identifier may be compatible with a standard protocol known as the Internationalized Resource Identifier (IRI) for example. An IRI may be understood as a string of characters that identifies a resource. An IRI may be a link to an internet based resource. For example, the symptom headache may correspond to a concept comprising the label “headache” and an identifier that is an IRI, and the IRI is linked by e.g. “http://health/test123”. The knowledge base stores simple concepts, each comprising a label and an identifier.

The Knowledge Base 150 comprises a network of medical concepts (diseases, drugs, treatments, risk factors, etc.) together with links described relations between them, e.g., <Headache treatedBy paracetamol>, <Malaria causedBy plasmodium>, <Malaria isA InfectiousDisease>. The links may be referred to as edges. The Knowledge Base 150 may therefore be represented using triples, comprising subject, property and object, <Subject, Property, Object>, each of these mapping to a concept and an IRI. An example of one triple would be <Breast cancer, isA, Cancer>. In one implementation, the Knowledge Base comprises 31,718,491 triples, with 8,310,814 concepts and 4 738 properties.

As described above, where the user input comprises free text, the diagnostic engine 111 calls one or more services to extract concepts from the text. For example a natural language parser which extracts the concepts which are present in the text and corresponding to one or more simple concepts in the knowledge base may be used. For example, a word2vec model may produce word embeddings, which are then mapped to concepts in the knowledge base. The diagnostic engine may then extract the concepts which correspond to concepts in the model used by the diagnostic engine 111. In other cases, the mapping may be pre-coded (for example given a question presented to the user, each answer corresponds to a concept used in the model).

The interface 105, diagnostic engine 111 and knowledge graph 150 may be located in a computing system, comprising a processor and memory, or in two or more computing systems.

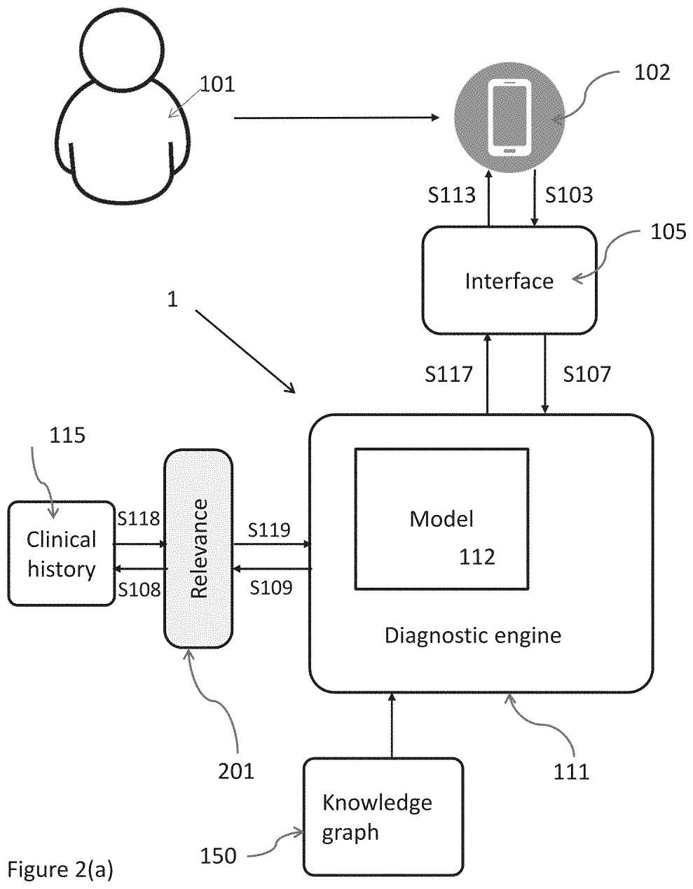

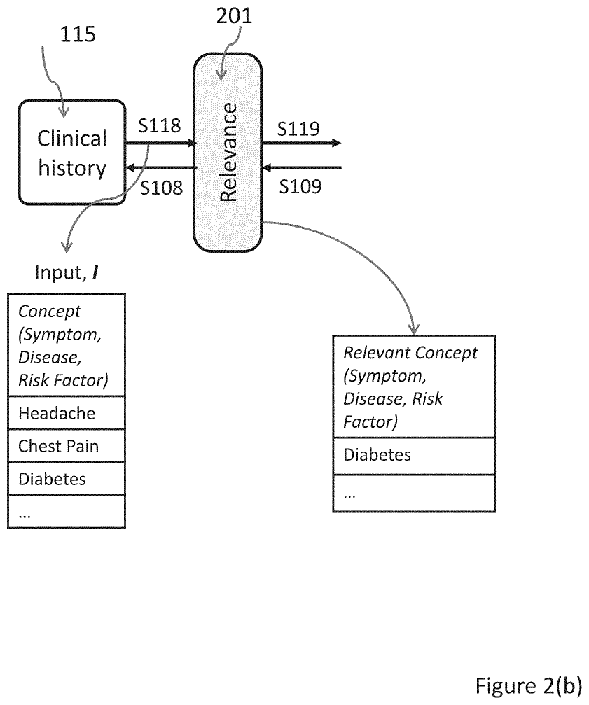

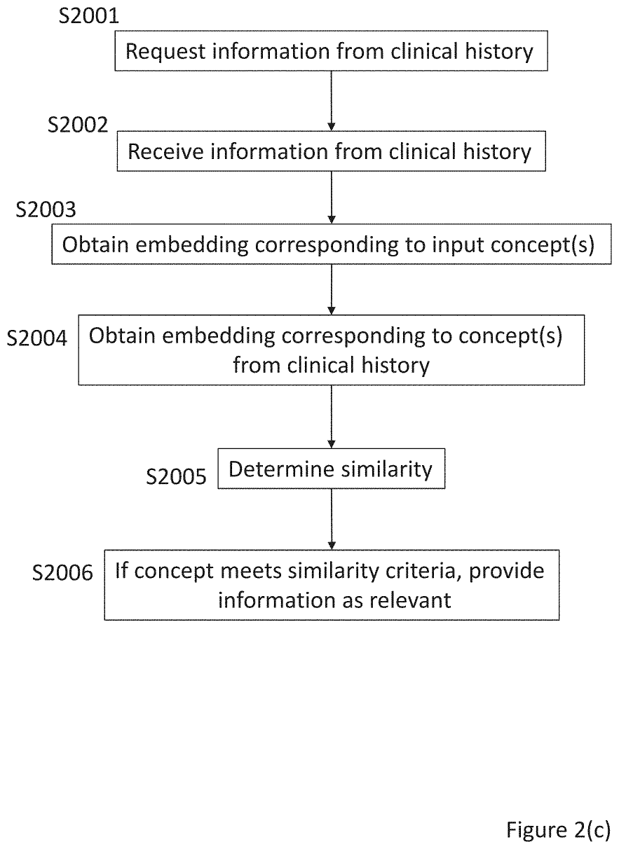

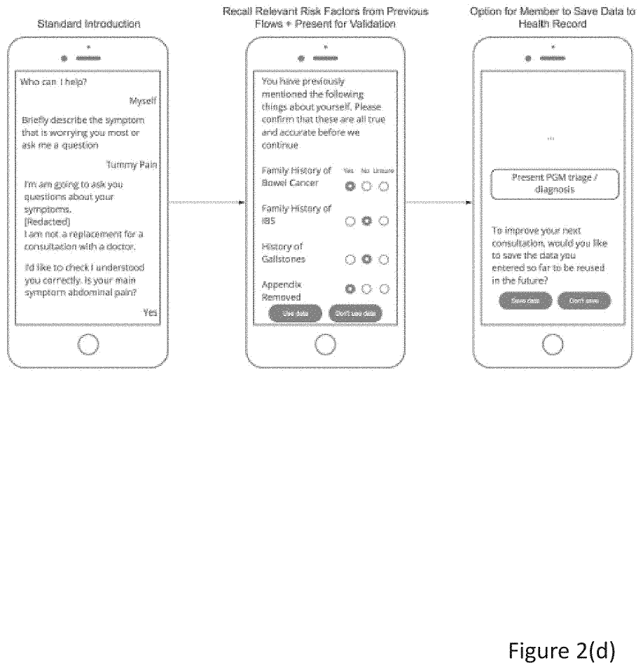

FIG. 2(a) is a schematic illustration of a medical diagnostic system 1 in accordance with an embodiment. For simplicity the same reference numerals are used for components that have been described previously in relation to FIG. 1. Description of the features which have been described previously is omitted. Again, a patient 101 communicates with the system via a device 102. The user terminal 102 communicates through the interface 105 as described previously in relation to FIG. 1(a).

To begin the diagnosis process, the patient 101 provides input comprising at least one symptom in step S103 via interface 105. This information is then sent to the diagnostic engine in step S107 in the same manner described previously. As described previously, the diagnostic engine 111 may extract a list of concepts which are present in the user input for example. This may correspond to a list of identifiers. It may also extract concepts which are used in the model and are identified by the user as absent.

In this embodiment, the diagnosis engine 111 is configured to identify the items of medical data, in this case symptoms, risk factors, and/or diseases, by their IRIs. Other types of identifiers may be used to represent these concepts, however, for simplicity, in the remainder of this specification we will refer to identifiers as IRIs.

The diagnostic engine 111 extracts the medical evidence (in this case at least one symptom which is identified as present, and optionally other items of medical data, and optionally those identified as absent) from the patient input.

The diagnostic engine 111 comprises a probabilistic model 112 from which the probability of one or more diseases being present can be calculated as has been described previously. From the calculated probabilities, the diagnosis engine 111 derives a likelihood that a disease is present and generates a diagnosis. The diagnosis may be output on the user terminal via steps S117 and S113. For example, for a given set of medical evidence (e.g. symptoms, diseases, or risk factors identified as present), if the diagnosis engine 111 calculates that P(disease=flu)=99%; P(disease=meningitis)=0.1%; and P(disease=measles)=0.2%, the diagnosis engine 111 will output that the disease is likely to be flu. The diagnosis may alternatively be output on another display device.

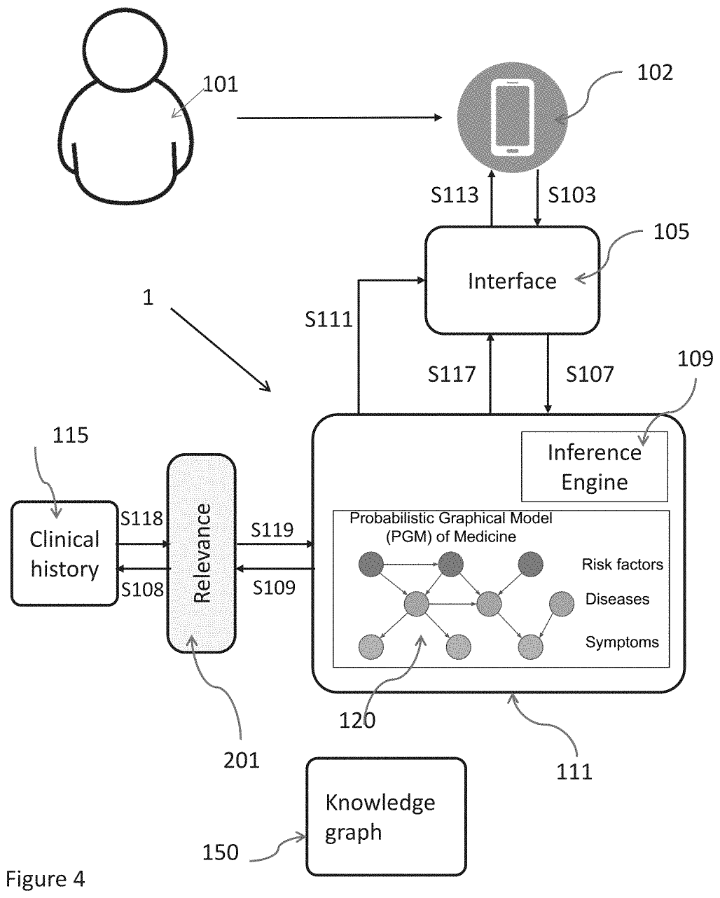

In an embodiment, the diagnosis engine 111 comprises an inference engine 109 and a PGM 120, as will be described in relation to FIG. 4 below. Other models may be used to perform the medical diagnosis however.

The probabilistic model 112 also takes as input relevant information relating to items of medical data (such as symptoms, risk factors and/or diseases) from a stored set of information relating to items of medical data (such as symptoms, risk factors and/or diseases) associated with the user. The stored set of information relating to items of medical data is stored in the clinical history 115.

In the following example, the items of medical data are symptoms, risk factors and/or diseases. However, other items of medical data, such as physiological data, recommendations or behaviours may also be included. Physiological data may include height, weight, body mass index (BMI), or VO2max for example. Recommendation may include medical advice such as “Do more exercise” or “Eat more vegetables”. Behaviour may include user behaviours such as “Physically active”, “Low physical activity”, or “Healthy eater”.

The diagnostic engine 111 first identifies the user corresponding to the received input. For example, the initial input from the user may comprise an HTTP request that contains a user ID and the text (comprising the at least one input symptom).

The diagnostic engine then calls for the set of the symptoms, risk factors and/or diseases which are to be used in the probabilistic model. The diagnostic engine sends this information to the relevance module 201, together with the information identifying the user and the user input. The set of the symptoms, risk factors and/or diseases used in the probabilistic model may comprise a list of IRIs corresponding to the concepts in the PGM for example. The user input comprises a list of IRIs corresponding to at least one symptom which is identified as present, and optionally diseases and/or risk factors, and optionally those identified as absent, from the patient input. The relevance module 201 then obtains the information relating to the user from the clinical history 112, based on the user identification.

This information from the clinical history may comprise one or more entries. Each entry may comprise an index, a patient ID, event information, the timestamp of the event, and the source of the event for example. The event information may comprise a concept which was identified as present or absent in the event. The events may include items of medical data from notes by a human doctor, prescriptions, user reported symptoms or events, healthchecks, lab tests, healthcare providers' databases or medical data from other sources. Events may be, for example, a diagnosis by the diagnostic engine or by a human doctor, a medical prescription, or an entry by a health monitoring service. The event information may further include information indicating whether the concept was identified as present (e.g. just the concept) or absent (e.g. the concept in combination with the NOT operator).

The relevance module 201 takes as input the information from the clinical history 115 of the user, and extracts a set of symptoms, risk factors and/or diseases used in the probabilistic model. The relevance module 201 determines the information corresponding to each of the set of symptoms, risk factors and/or diseases used in the probabilistic model from the stored information associated with the user (for example whether the symptoms, risk factors and/or diseases are present, absent or this is unknown in this clinical history).

The relevance module then determines a measure of relevance of each concept to the medical evidence (in this case at least one symptom which is identified as present, and optionally diseases and/or risk factors, and optionally those identified as absent) from the patient input. Specifically, for each concept in the patient input, the relevance module 201 determines a measure of relevance for each concept extracted from the clinical history 115. Examples of how this relevance may be determined are described below, in relation to FIGS. 2(b) and (c).

FIG. 2(b) is a schematic illustration of a module 201 for determining the relevance of medical information. The relevance module 201 may be a relevance module used in a medical diagnosis system in accordance with an embodiment, such as that described in relation to FIG. 2(a) above for example. Alternatively, it may be part of a different system, or may be a separate system for determining the relevance of medical information.

The relevance module 201 obtains information regarding items of medical data associated with a user and stored in a clinical history 115. It determines a measure of relevance of the items, e.g. symptoms, risk factors and/or diseases, to one or more target items. It may then provide the relevant information as output.

In the below described examples, the information corresponds to information indicating whether the concept is present or absent. However, the system may only use “present” for example. Alternative information may be provided.

The relevance module 201 obtains information from a stored clinical history 115, which comprises medical data about a patient. The clinical history 115 may be a database stored within the same system as the relevance module 201, or it may be located remotely in a separate system. The clinical history comprises a record of information relating to items of medical data (i.e., symptoms, diseases, and/or risk factors). These may be those previously reported by a diagnosis engine or notes from a human doctor, prescriptions, user reported symptoms or events, or medical data from other sources, as described above for example.

The relevance module 201 may obtain the information from the stored clinical history 115 in response to some input. This input may be a request for information relating to a set of symptoms, risk factors and/or diseases comprised in a model used by a medical diagnosis system, as described in the example relating to FIG. 2(a) above. Alternatively, it may be simply a request for the valid clinical history from e.g. a doctor or user.

FIG. 2(c) shows a flow chart illustrating a method of determining relevance, which is performed as part of a method of medical diagnosis in accordance with an embodiment. For example, in step S108 shown in FIG. 2(a), the relevance module 201 requests information from the patient's clinical history 115. This corresponds to S2001 in FIG. 2(c). The clinical history service provides information to the relevance module 201 in step S118 of FIG. 2(a). This corresponds to S2002 in FIG. 2(c). The information provided to the relevance module 201 may comprise a list of concepts. The information may indicate whether the concept is present (for example by including the concept) or absent (for example by including the operator “NOT” together with the concept). Alternatively, the information may simply indicate that the concept is present.

In S2003, the relevance module 201 obtains an embedding, i.e. a vector, for each target concept. In a method of medical diagnosis, the target concepts are those in the patient input, sent from the diagnosis engine 111 to the relevance module in S109 of FIG. 2(a). The patient input comprises at least one concept corresponding to a symptom which is identified as present. In the below, the case will be described where the patient input comprises only one concept corresponding to a symptom. However, the patient input may comprise multiple concepts, corresponding to more than one symptom, and/or diseases and/or risk factors, and optionally those identified as absent as well as those identified as present. Other target concepts may be used.

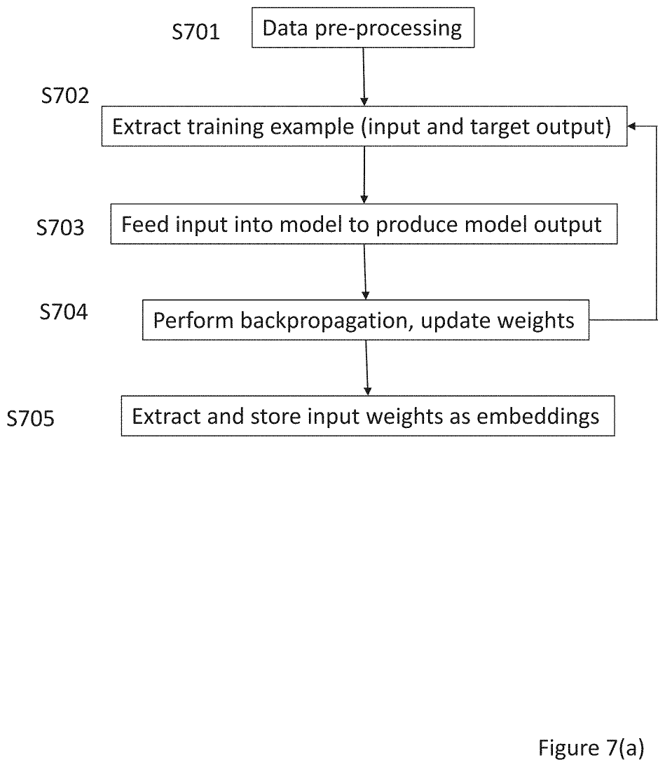

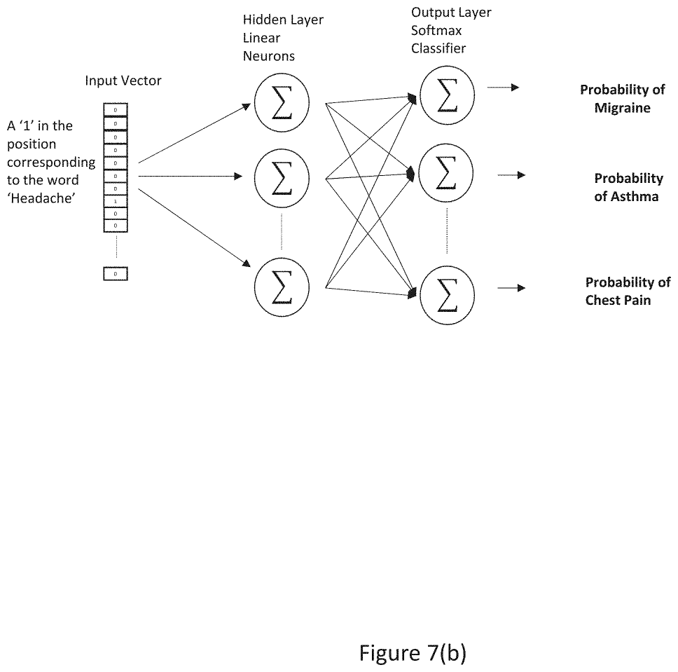

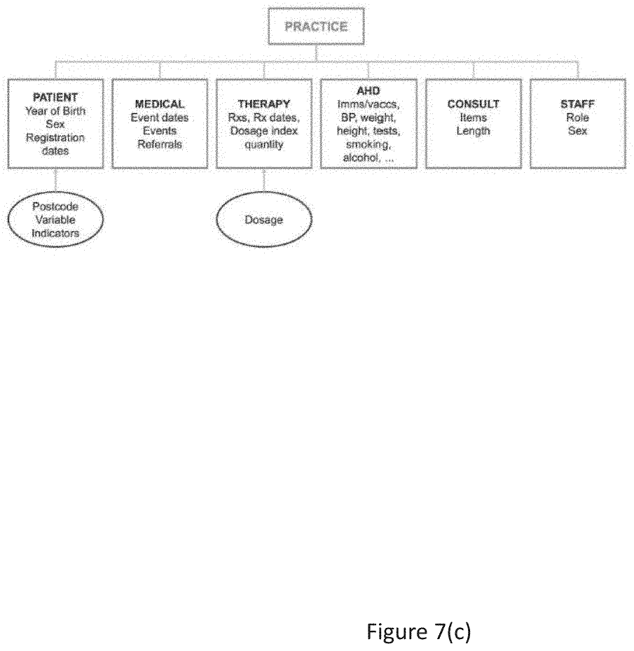

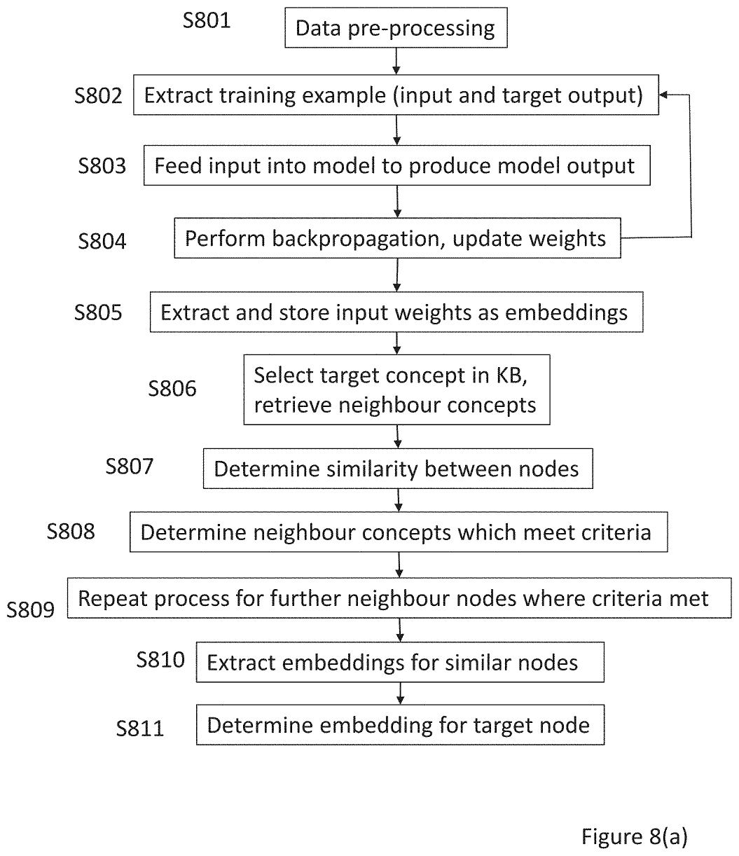

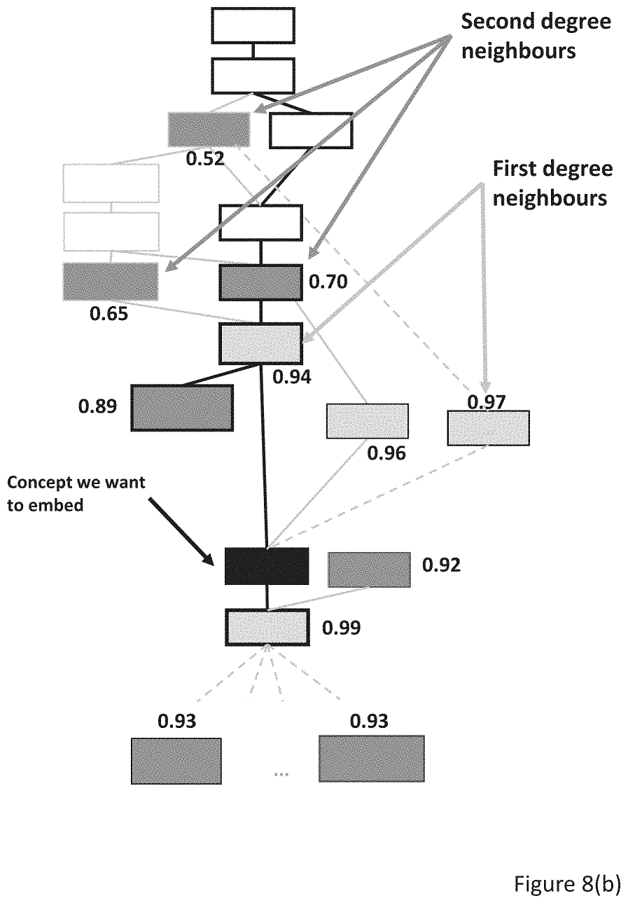

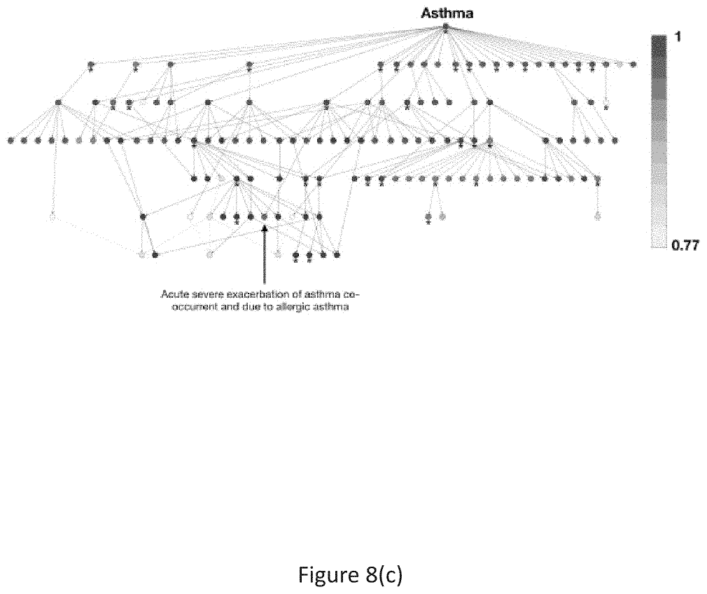

The embedding corresponding to the input symptom is retrieved from a stored set of embeddings. The embeddings are generated in a training stage. Example training methods in which the embeddings are generated are described below in relation to FIGS. 7 and 8. Once generated, they are stored and can then be retrieved by the relevance module 201 during operation. The embeddings may be stored in a common system with the relevance module 201, or may be stored in a separate system and retrieved from the separate system when needed. The dimensions for the embeddings n may be selected as a hyperparameter during training. In an embodiment, the embedding vectors have length of 128. Alternatively, a length of 32 or 512 could be used for example. A length of between 50 and 200 was found to result in a good precision during testing, as will be described below.

In S2004, the relevance module 201 obtains an embedding for each concept extracted from the clinical history, in the same manner.

In S2005, a similarity measure between each concept in the clinical history and the input concept is calculated. Various methods of calculating a similarity measure between two vectors may be used. In an embodiment, the cosine similarity is used:

$$similarity = \frac{A . B}{‖ A ‖ . ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}}$$

For a vector corresponding to an input concept A, for each vector corresponding to a concept in the clinical history B, a cosine similarity is calculated. In above, n is the dimension of the vectors. In an embodiment, n=128.

Thus at S2005, for each concept extracted from the clinical history for the patient, a corresponding similarity value is generated. Although the cosine similarity is described here as an example, other measures of similarity between two vectors may be used, for example a Euclidean distance. Cosine similarity values range from −1 to 1, with values closer to 1 implying higher similarity, and values closer to −1 implying higher dissimilarity. The score may be scaled to a range of 0 to 1 for example, with 0 implying maximum dissimilarity and 1 implying maximum similarity. The similarity measure is an example of a measure of relevance.

In S2006, the measure of relevance corresponding to the concepts from the clinical history are used to determine a sub-set of relevant concepts. The sub-set of relevant concepts may be a pre-determined number of concepts having the highest similarity values. For example, the concepts corresponding to the highest 10 similarity values may be selected as the relevant concepts, and the information from the clinical history relating to these 10 concepts (for example whether the concept is present or absent) is provided as relevant information in S109 of FIG. 2(a). Alternatively, the concepts having a similarity value higher than a pre-determined threshold may be selected as the relevant concepts. In an embodiment, the threshold is 0.9 for example, where the similarity ranges between 0 and 1.

The above has been described for the case where the patient input comprises a single concept. Where the patient input comprises multiple concepts, for each concept in the clinical history, a similarity value is calculated for each concept in the patient input. An average value is then taken as the similarity value for the concept from the clinical history. Whether the information relating to the concept in the clinical history is relevant is determined in the same manner as described above, using the average similarity value.

The relevance module 201 may process simple concepts such as headache, but may also operate on complex concepts (e.g. Severe Headache). In this case, an embedding may be obtained for a base simple concept (for example “Heacache”), and this may be used to determine the measure of relevance. Alternatively, an embedding may be generated corresponding to one or more complex concepts in the training stage, for example if these are contained in the training dataset, and stored.

The relevance module 201 scores one or more entries in the medical history of a given user based on their relevance to an input concept or concepts, as shown in the example below:

Map<IRI , Float> calculateMemberRelevance (Id memberId, Set<IRI> presentingConcepts ) {

return calculateHistoryRelevance (getHistory (memberId) , presentingConcepts)

}
Map<IRI , Float> calculateHistoryRelevance (Set<IRI> medicalHistoryOfMember, Set<IRI>
presentingConcepts) ;

It takes as input a set of one or more presenting concepts and the medical history of a given user. In an embodiment, it outputs a score∈[0, 1] for each item in the medical history, showing the relevance of that item to the set of input concepts, where 0 indicates the lowest likelihood of relevancy and 1 the highest likelihood of relevancy. A concept may be considered as ‘relevant’ if the score assigned is over a threshold for example.

The clinical history 115 of the user may comprise information from a doctor's note for example, indicating that the patient has diabetes. The information about symptoms, risk factors and/or diseases from the clinical history 115 of the user therefore comprises an indication that the disease “diabetes” is present. If this information is determined to be relevant, the relevance module 201 may generate an entry “1” corresponding to the IRI representing the concept “diabetes”. Similarly, the clinical history 115 of the user may comprise information from a doctor's note for example, indicating that the patient does not have “asthma”. If this information is determined to be relevant, the relevance module 201 may generate an entry “−1” corresponding to the IRI representing the concept “asthma”. If it is unknown whether the symptom, risk factor and/or disease is present or absent, this may be indicated by a 0. Similarly, if the symptom, risk factor and/or disease is determined not to be relevant, a 0 is used. As previously stated, other means of indicating this information may be used however.

By comparing the symptoms, risk factors and/diseases from the diagnostic engine to the information about the relevant symptoms, risk factors and/or diseases from the clinical history, information about the relevant symptoms, risk factors and/or diseases used in the model is obtained (for example, whether each is present, or absent).

Thus the relevance module 201 first retrieves information relating to items of medical data from the stored clinical history 115, and then determines the relevance of each item of medical data corresponding to the set of symptoms, risk factors and/or diseases to be used in the probabilistic model. This may comprise determining which of the symptoms, risk factors and/or diseases which are indicated as being present or absent in the clinical history 115 are relevant to the patient input information. The relevance module 201 then passes information relating to the relevant symptoms, risk factors and/or diseases back to the diagnostic engine 111. This information is also taken as input to the probabilistic model 112 (as well as the information from the user input). The probabilistic model 112 determines the probability of the user having each of one or more diseases from the input information.

The diagnostic engine 111 is therefore configured to retrieve further information in step S109 from the stored clinical history, in addition to the information provided by the user in S107. The diagnostic engine 111 retrieves this information by calling a service called a “relevance” service 201 (also referred to as the relevance module 201). The relevance module 201 acts as an interface between the diagnostic engine 111 and the patient's stored clinical history 115 that pre-processes information for items of medical data stored in the clinical history.

The relevance module 201 may output the set of IRIs representing the relevant symptoms, risk factors and/or diseases used in the probabilistic model, together with the information (for example, the information may indicate, for each concept in the model, whether the concept is present or absent). This may be combined with the information received from the user and input into the model 112. The combination may be performed according to some pre-defined priority, as will be described below.

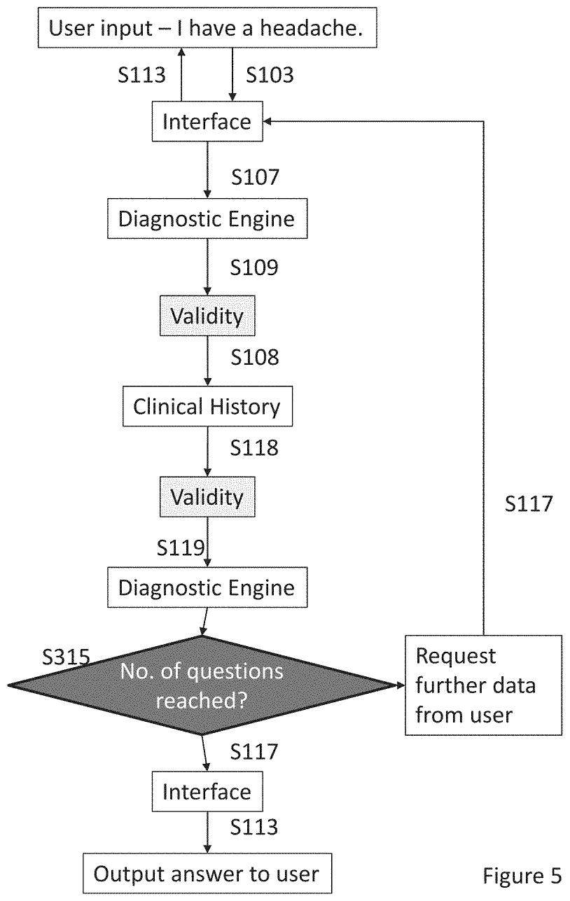

The probabilistic model 112 takes as input evidence, for example the presence or absence of various symptoms, diseases and/or risk factors, from the clinical history and the information received from the user. For symptoms and risk factors where the patient has been unable to provide a response, and for which the status is unknown in the clinical history, or which are not determined to be relevant, these are initially assumed to be unknown. As will be described in relation to FIG. 5 below, the user and clinical history may only provide partial information, and therefore the system can be adapted to request further information from the patient. With this approach, several rounds of dialog between the user 101 and the diagnostic system 111 may be used to obtain information to make an accurate diagnosis. It is desirable that the questions asked of the user are more relevant and thus lead to a more accurate diagnosis.

In an embodiment, some or all of the information obtained from the relevance module 201 is confirmed with the user before being input to the model 112. For example, some or all of the information output from the relevance module 201 as being relevant may be sent to the user through the user interface, and the user requested to confirm the information is correct, and correct the information if incorrect. The confirmed and/or corrected information received from the user is then sent back to the diagnostic engine and the input evidence generated from the confirmed/corrected information.

Retrieving information from the clinical history 115 may provide additional evidence that was not reported by the user in the input. The user clinical history is likely to comprise a large data set however. Some of the information derived from the clinical history 115 may not be relevant. If irrelevant information is entered in the diagnosis engine 111 of FIG. 2(a), this can result in less accurate diagnosis in some cases. Furthermore, if irrelevant information is included, this results in transmission of a large amount of data to the user device for confirmation.

The data may therefore be pre-processed, before inputting into the model, so that information determined to be relevant is inputted. The system pre-processes the information from the stored information to identify a sub-set of relevant information. A more accurate diagnosis may be obtained in some cases, and less data is required to be transmitted.

The clinical history may also be referred to as the user graph. The user graph is a health record, comprising the information collected by different services about a patient. For each interaction that the patient has with the system, and optionally other services, the collected information is stored into a Clinical History and the data is converted as medical concepts coded in the Knowledge Graph. The relevance module 201 allows filtering of the information to exclude less useful data points. The user graph mitigates a need to re-ask the same questions to the patient at every encounter. It can also give more context to the diagnosis system to allow improvement of the diagnosis.

The diagnosis model is integrated with the User Graph (clinical history), providing the diagnosis mode with a “memory”. Instead of each diagnosis flow (e.g. PGM flow) being independent and agnostic of all others, each flow is able to recall the status of previous diseases and disease risks gathered from previous flows carried out on the same user. However, if the User Graph simply provides all known prior medical information about the user (or even all valid concepts, as described below), large amounts of data must be transmitted, and a less accurate diagnosis may be provided in some cases.

FIG. 4 is a schematic illustration of a medical diagnostic system 1 in accordance with an embodiment. The diagnostic engine 111 comprises an inference engine 109 and a probabilistic graphical model (PGM) 120. Although an embodiment in which a PGM is used is described here, other models can alternatively or additionally be used, for example one or more neural networks.

In the system shown in FIG. 4, and as described previously, follow-up questions may be asked by the interface 105. How this is achieved will be explained later. First, it will be assumed that there are no follow-up questions. This will be used to explain the basic procedure. However, a variation on the procedure will then be explained where the diagnosis engine, once completing the first analysis, requests further information.

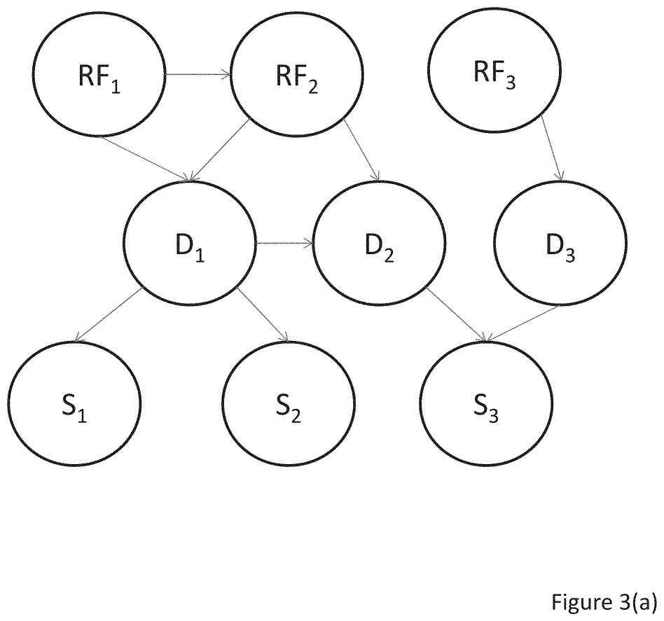

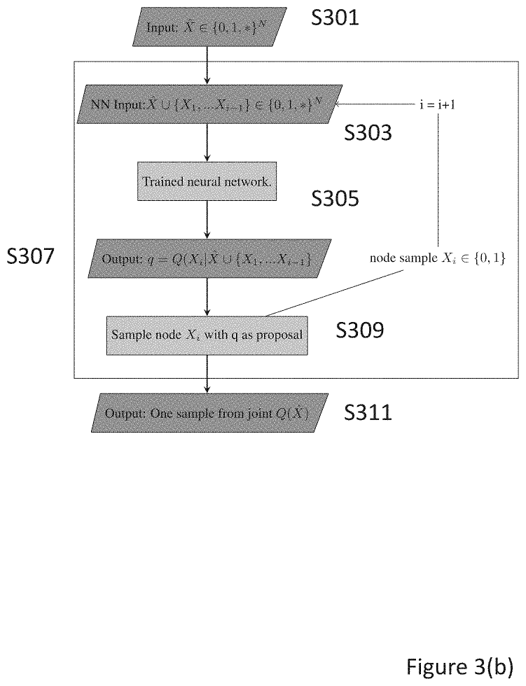

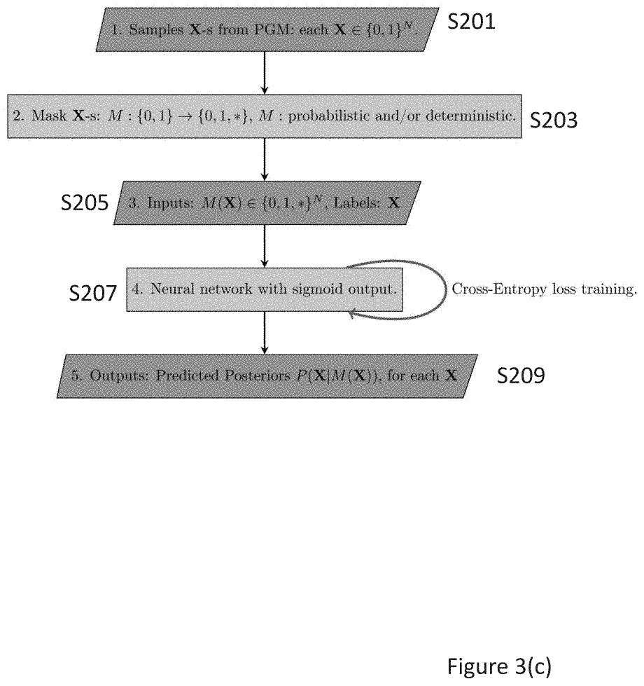

Inference engine 109 performs Bayesian inference on PGM 120. PGM 120 will be described in more detail with reference to FIG. 3(a). In the medical diagnosis system, performing inference on the PGM provides the likelihood of a set of diseases, based on the evidence provided.

The inference engine 109 calculates “likelihood” (conditional marginal probability) P(Disease_i|Evidence) for all diseases.

In addition the inference engine can also determine:

P(Symptom_i|Evidence),

P(Risk factor_i|Evidence).

From this, it can transmit back information in step S117 concerning the “likelihood” of a disease—in other words, a diagnosis—given the evidence supplied by the patient 101 and the clinical history. The interface 105 can then supply this information back to the mobile phone 102 of the patient in step S113.

Due to the size of the PGM 120, it may not be possible to perform exact inference using inference engine 109 in a realistic timescale. Instead, the inference engine 109 may perform approximate inference. The inference engine may be configured to perform approximate inference using importance sampling over conditional marginals. However, other methods may be used such as variational inference, other Monte Carlo methods, etc.

Approximate inference may comprise sampling from an independent ‘proposal’ distribution, which ideally is as close as possible to the target (true posterior distribution). The inference engine uses an approximation of the outputs of the probabilistic graphical model as proposals for subsequent sampling. One approach when applying Bayesian networks for medical decision-making is to use the model prior as the proposal distribution. Other approaches can be used, for example generating a proposal distribution using a neural network. An example of this approach will be described below, however various other methods may be used.

Inference may be performed by considering the set of random variables, X=(X₁, . . . X_{N}). A BN is a combination of a directed acyclic graph (DAG), with Xᵢ as nodes, and a joint distribution of the Xᵢ, P. The nodes Xᵢ correspond to the risk factors, symptoms and diseases as shown in FIG. 3 (a) for example, and described in more detail below. The distribution P can factorize according to the structure of the DAG:

$$P ( {X}_{1} … , {X}_{n} ) = {\prod }_{i = 1}^{N} P ( {X}_{i} \vert Pa ( {X}_{i} ) ) = P ( {X}_{1} ) {\prod }_{i = 2}^{N} P ( {X}_{i} \vert {X}_{1} , … , {X}_{i - 1} ) , \qquad ( 1 )$$

Where P(Xᵢ|Pa(Xᵢ)) is the conditional distribution of Xi given its parents, Pa(Xᵢ). The second equality holds as long as X₁; X₂; : : : ; X_{N} are in topological order.

Now, a set of observed nodes is considered, [symbol] ⊂X and their observed values \hat{x}. To conduct Bayesian inference when provided with a set of unobserved variables, say Xᵤ⊂X\ [symbol] , the posterior marginal is computed:

$$P ( {X}_{𝒰} \vert {X}_{𝒪} = \hat{x} ) = \frac{P ( {X}_{𝒰} , {X}_{𝒪} = \hat{x} )}{P ( {X}_{𝒪} = \hat{x} )} = \frac{P ( {X}_{𝒰} ) P ( {X}_{𝒪} - \hat{x} \vert {X}_{𝒰} )}{P ( {X}_{𝒪} = \hat{x} )} \qquad ( 2 )$$

In the optimal scenario, Equation (2) could be computed exactly. However, as noted above exact inference becomes intractable in large BNs as computational costs grow exponentially with effective clique size,—in the worst case, becoming an NP-hard problem.

In an embodiment, importance sampling is used to perform approximate inference. Here, a function f is considered for which its expectation, Ep[f] is to be estimated, under some probability distribution P. It is often the case that we can evaluate P up to a normalizing constant, but sampling from it is costly.

In Importance Sampling, expectation Ep[f] is estimated by introducing a distribution Q, known as the proposal distribution, which can both be sampled and evaluated. This gives:

$$\begin{matrix}{E}_{p} [ f ] = \int f ( x ) P ( x ) dx \\ = \int f ( x ) \frac{P ( x )}{Q ( x )} Q ( x ) dx \\ = {lim}_{n \to ∞} \frac{1}{n} {\sum }_{i = 1}^{n} f ( {x}_{i} ) {w}_{i} ,\end{matrix} \qquad ( 3 )$$

Where xᵢ˜Q and where wᵢ=P(xᵢ)/Q(xᵢ) are the importance sampling weights. If P can only be evaluated up to a constant, the weights need to be normalized by their sum.

In the case of inference on a BN, the strategy is to estimate P(Xᵤ| [symbol] ) with an importance sampling estimator if there is appropriate Q to sample from. One approach when applying Bayesian networks for medical decision-making is to use the model prior as the proposal distribution Q.

Alternatively, a further model may be used to generate a proposal distribution corresponding to a joint distribution Q=P(Xᵤ| [symbol] ). For example, the evidence may be passed to a universal marginaliser (UM). A UM is a neural network that has been trained to approximate the outputs of the PGM, for example a single feedforward neural net, or a neural network which comprises several sub-networks (such that the whole architecture is a form of auto-encoder-like model but with multiple branches). The UM returns probabilities to be used as proposals to the inference engine, based on the input evidence. The inference engine then performs importance sampling using the proposals from the UM as estimates.

An example inference method using a Universal Marginalizer has been described in the document Douglas, L., Zarov, I., Gourgoulias, K., Lucas, C., Hart, C., Baker, A., Sahani, M., Perov, Y. and Johri, S., 2017, A Universal Marginalizer for Amortized Inference in Generative Models, arXiv preprint arXiv:1711.00695, which is incorporated herein by reference.

The UM (e.g. a feedforward neural network) may be trained by sampling from the PGM. An example training process for the above described UM involves generating samples from the underlying BN, in each sample masking some of the nodes, and then training with the aim to learn a distribution over this data. An example training process using this approach is illustrated in FIG. 3(c).

The UM model is trained off-line by generating samples from the original BN (PGM) via ancestral sampling in step S201. In an embodiment, unbiased samples are generated from the probabilistic graphical model (PGM) using ancestral sampling. Each sample is a binary vector which will be the values for the classifier to learn to predict.

For the purpose of prediction, some nodes in the sample may then be hidden, or “masked” in step S203. This masking is either deterministic (in the sense of always masking certain nodes) or probabilistic over nodes. In embodiment each node is probabilistically masked (in an unbiased way), for each sample, by choosing a masking probability p˜U[0,1] and then masking all data in that sample with probability p.

The nodes which are masked (or unobserved when it comes to inference time) are represented consistently in the input tensor in step S205. Different representations of obscured nodes will be described later, for now, they will be represented as a ‘*’.

The neural network is then trained using a cross entropy loss function in step S207 in a multi-label classification setting to predict the state of all observed and unobserved nodes. The output of the neural net can be mapped to posterior probability estimates. Any reasonable, i.e., a twice-differentiable norm, loss function could be used. However, when the cross entropy loss is used, the output from the neural net is exactly the predicted probability distribution.

The trained neural network can then be used to obtain the desired probability estimates by directly taking the output of the sigmoid layer. This result could be used as a posterior estimate.

However, the output approximation can also be used as a proposal for any inference method (e.g. as a proposal for Monte Carlo methods or as a starting point for variational inference, etc). An example method of importance sampling is described in relation to FIG. 3(b).

In the above discussion of Importance sampling, we saw that the optimal proposal distribution Q for the whole network is the posterior itself P(Xᵤ| [symbol] ), and thus for each node the optimal proposal distribution Qₒₚₜ=P(Xᵢ∈Xᵤ| [symbol] ∪X_{S}), where [symbol] are the evidence nodes and X_{S} the already sampled nodes before sampling Xᵢ.

As it is now possible using the above UM to approximate, for all nodes, and for all evidences, the conditional marginal, the sampled nodes can be incorporated into the evidence to get an approximation for the posterior and use it is as proposal. For node i specifically, this optimal Q* is:

Qᵢ*=P(Xᵢ|{X₁, . . . ,Xᵢ₋₁}∪ [symbol] =\hat{x})≈UM({X₁, . . . ,Xᵢ₋₁}∪ [symbol] )ᵢ=Qᵢ (5)

The process for sampling from these approximately optimal proposals is illustrated in the algorithm 1 below and in FIG. 3(b) where the part within the box is repeated for each node in the BN in topological order.

In step S301, the input is received and passed to the UM (NN). The NN input is then provided to the NN (which is the UM) in step S303. The UM calculates in step S305, the output q that it provides in step S307. This is the provided to the Inference engine in step S309 to sample node Xᵢ from the PGM. Then, that node value is injected as an observation into \hat{x}, and it is repeated for the next node (hence ‘i:=i+1’). In step S311, we receive a sample from the approximate joint.

Algorithm 1 Seguential Universal Marginalizer importance sampling

| | |
|---|---|
| 1: | Order the nodes topologically X₁, . . . X_{N}, where N is the total number of nodes. |
| 2: | for j in [1, . . . , M] (where M is the total number of samples): do |
| 3: | [symbol] = ∅ |
| 4: | for i in [1, . . . N]: do |
| 5: | sample node xᵢ from Q(Xᵢ) = UM( [symbol] )ᵢ ≈ P(Xᵢ\| [symbol] , [symbol] ) |
| 6: | add xᵢ to [symbol] |
| 7: | [symbol] = [symbol] |
| 8: | ${w}_{j} = {\prod }_{i = 1}^{N} \frac{{P}_{i}}{{Q}_{i}} ( where {P}_{i} is the likelihood , {P}_{i} = P ( {X}_{i} = {x}_{i} \vert {x}_{𝒮 ⋂ Pa ( {X}_{i} )} and$ |
| | Qᵢ = Q(Xᵢ = xᵢ)) |
| 9: | ${E}_{p} [ X ] = \frac{{\sum }_{j = 1}^{M} {X}_{j} {w}_{j}}{{\sum }_{j = 1}^{M} {w}_{j}} ( as in standard IS )$ |

That is, following the requirement that parents are sampled before their children and adding any previously sampled nodes into the evidence for the next one, we are ultimately sampling from the approximation of the joint distribution. This can be seen by observing the product of the probabilities we are sampling from.

It can be seen that the proposal Q, constructed in such a way, becomes the posterior itself:

$$\begin{matrix}Q = {\prod }_{i = 1}^{N} {Q}_{i} & ( 6 ) \\ = {UM ( {X}_{𝒪} )}_{i} {\prod }_{i = 2}^{N} {UM ( {X}_{1} , … , {X}_{i - 1} , )}_{i} & ( 7 ) \\ \approx P ( {X}_{1} \vert {X}_{𝒪} ) {\prod }_{i = 2}^{N} P ( {X}_{i} \vert {X}_{1} , … , {X}_{i - 1} , {X}_{𝒪} ) & ( 8 ) \\ = P ( {X}_{1} , {X}_{2} , … , {X}_{n} \vert {X}_{𝒪} ) & ( 9 )\end{matrix}$$

This procedure requires that nodes are sampled sequentially, using the UM to provide a conditional probability estimate at each step. This can affect computation time, depending on the parallelization scheme used for sampling. However, parallelization efficiency can be recovered by increasing the number of samples, or batch size, for all steps.

In Importance Sampling, each node will be conditioned on nodes topologically before it. The training process may therefore be optimized by using a “sequential masking” process in the training process as in FIG. 3(c), where firstly we randomly select up to which node Xᵢ we will not mask anything, and then, as previously, mask some nodes starting from node Xᵢ₊₁ (where nodes to be masked are selected randomly, as explained before). This is to perform to a more optimal way of getting training data.

Another approach might involve a hybrid approach as shown in Algorithm 2 below. There, an embodiment might include calculating the conditional marginal probabilities only once, given the evidence, and then constructing a proposal for each node Xᵢ as a mixture of those conditional marginals (with weight β) and the conditional prior distribution of a node (with weight (1−β)).

Algorithm 2 Hybrid UM-IS

| | |
|---|---|
| 1: | Order the nodes topologically X₁, . . . X_{N}, where N is the total number of nodes. |
| 2: | for j in [1, . . . , M] (where M is the total number of samples): do |
| 3: | [symbol] = ∅ |
| 4: | for i in [1, . . . N]: do |
| 5: | sample node xᵢ from Q (Xᵢ) = βUMᵢ( [symbol] ) + (1 − β)P(Xᵢ = xᵢ\| [symbol] ) |
| 6: | add xᵢ to [symbol] |
| 7: | [symbol] = [symbol] |
| 8: | ${w}_{j} = {\prod }_{i - 1}^{N} \frac{{P}_{i}}{{Q}_{i}} ( where {P}_{i} is the likelihood , {P}_{i} = P ( {X}_{i} = {x}_{i} \vert {x}_{𝒮 ⋂ Pa ( {X}_{i} )} and$ |
| | Qᵢ = Q(Xᵢ = xᵢ)) |
| 9: | ${E}_{p} [ X ] = \frac{{\sum }_{j = 1}^{M} {X}_{j} {w}_{j}}{{\sum }_{j = 1}^{M} {w}_{j}} ( as in standard IS )$ |

While this hybrid approach might be easier and potentially less computationally expensive, in cases when P(Xᵢ|X_{S}∪ [symbol] ) is far from P(Xᵢ| [symbol] ), this will be just a first-order approximation, hence the variance will be higher and we generally need more samples to get a reliable estimate.

The intuition for approximating P(Xᵢ| [symbol] ∪X_{S}) by linearly combining P(Xᵢ|Pa(Xᵢ)) and UM( [symbol] )ᵢ is simply that UM( [symbol] )ᵢ will take into account the effect of the evidence on node i and P(Xᵢ|Pa(Xᵢ)) will take into account the effect of X_{S}, namely the parents. Note that β could also be allowed to be a function of the currently sampled state and the evidence, for example if all the evidence is contained in parents then=0 is optimal.

Although the above describes an inference method using importance sampling, alternatively, the sampling step may be omitted, and the trained UM can be used to directly approximate the posterior. The output of the UM comprises a vector of conditional marginal probabilities for every node in the BN, whether observed or not (if node Xᵢ is observed, the marginal posterior distribution for it will be trial, i.e. P(Xᵢ| [symbol] )=1 or P(Xᵢ| [symbol] )=0). The probabilities corresponding to the disease nodes can then be used directly for diagnosis, omitting the sampling step.

Although one method of performing inference has been described above, other models can be used to generate P(Disease_i|Evidence) for all diseases.

FIG. 3(a) is a depiction of a graphical model of the type used in the system of FIG. 4 according to an embodiment. The graphical model provides a natural framework for expressing probabilistic relationships between random variables, to facilitate causal modelling and decision making. In the model of FIG. 3(a), when applied to diagnosis, D stands for disease, S for symptom and RF for Risk Factor. The model therefore has three layers: risk factors, diseases and symptoms. Risk factors (with some probability) influence other risk factors and diseases, diseases cause (again, with some probability) other diseases and symptoms. There are prior probabilities and conditional marginals that describe the “strength” (probability) of connections.

In this simplified specific example, in the first layer, there are three nodes S₁, S₂ and S₃, in the second layer there are three nodes D₁, D₂ and D₃ and in the third layer, there are two nodes RF₁, RF₂ and RF₃. Each arrow indicates a dependency. For example, D₁ depends on RF₁ and RF₂. D₂ depends on RF₂ and D₁. Further relationships are possible. In the graphical model shown, each node is only dependent on a node or nodes from a different layer. However, nodes may be dependent on other nodes within the same layer.

In an embodiment, the graphical model of FIG. 3(a) is a Bayesian Network (BN). The network represents a set of random variables and their conditional dependencies via a directed acyclic graph. Thus, in the network of FIG. 3(a), given full (or partial) evidence over symptoms S₁, S₂ and S₃ and risk factors RFᵢ, RF₂ and RF₃ the network can be used to represent the probabilities of various diseases D₁, D₂, and D₃.

In summary, the PGM 120 captures the probabilistic relationship between entities such as risk-factors, diseases, and symptoms. Given a set of evidence, the inference engine 109 performs Bayesian inference from the PGM 120 and calculates a “likelihood” (conditional marginal probability) of a disease given a set of evidence for all diseases. Performing exact inference is computationally expensive and approximations are generally used to speed up the computation. Example methods have been described above for performing this calculation.

Information about the concepts (e.g. S₁, S₂ and S₃, RF₁, RF₂ and RF₃ and D₁, D₂, and D₃ in the example) is obtained from the user input in steps S₁₀₃ and S₁₀₇, and from the clinical history in S119. How the information from both sources is combined will be described later. This information corresponds to the nodes in the PGM, and is taken as the input evidence into the inference engine (i.e. the observed nodes).

The value of the state of the node reflects whether the concept represented by the node is true (1)—that is, it should have an impact on the calculation of probabilities—or false (0)—that is, it should not have any bearing on the calculation. It will be understood that the state of a node can have a value that is not restricted to 0 or 1.

As described above, the relevance module 201 may output information for each relevant concept in the clinical history indicating whether it is present (1) or absent (−1). This information is combined with the user input. The input to the model consists of the presence (1) or absence (0) of two or more concepts in the model. An output of 1 from the relevance module 201 is mapped to “1”, an output of −1 from the relevance module is mapped to “0”.

For example, the input evidence may comprise a vector \hat{x}, in which each entry corresponds to a node of the PGM. In the example described above, in which the inference engine uses importance sampling together with a universal marginaliser, the input evidence vector \hat{x} is provided as input to the universal marginaliser (comprising a neural network). The output from the universal marginaliser is then provided to the inference engine to sample node Xᵢ (corresponding to a disease) from the PGM. Then, that node value is injected as an observation into \hat{x}, and it is repeated for the next node. Finally, a sample is received from the approximate joint distribution.

Alternatively, the sampling step may be omitted, the input evidence vector \hat{x} provided as input to the trained neural network, the output of the neural network being directly used as the probability of each node corresponding to a disease.

As described above, the input medical data vector \hat{x} may comprise an entry corresponding to each node in the PGM, where the entry is a 1 if the symptom, risk factor or disease is indicated as present (as determined from the information in the user input and the output from the relevance module 201), and the entry is a 0 if the symptom, risk factor or disease is absent (as determined from the information in the user input or in the output from the relevance module 201). A value of 0.5 may be assigned where the presence/absence is unknown.

Where there is a conflict between the user input and the clinical history information, or where the clinical history information itself comprises a conflict, a pre-defined priority may be used to select the information used. This will be described below.

Using information from the clinical history 115 may allow the presence (and optionally absence) of a greater number of nodes to be determined initially. Having values for more of the nodes enables the diseases to be computed with greater certainty (higher probability). In other words, entering information about a larger number of concepts, e.g. by having access to a clinical history, into the diagnosis engine 111 enables the diagnosis engine to determine diseases with more certainty (higher probability) and arrive at an accurate diagnosis.

However, if a large amount of information is entered but some of it is not relevant, this may result in a less accurate diagnosis in some cases. The values of the nodes corresponding to different concepts (risk factors, disease or symptom) taken as input to the probabilistic model 112 affect the values of the computed probabilities, and therefore the accuracy of the diagnosis. By pre-processing the information from the clinical history 115, before inputting it into the model 112, only information determined to be relevant is input to the model 112.

In the example described above, according to an embodiment, the diagnostic engine 111 is configured to take information from the interface in S107, where the input is converted to a list of as of the concepts reported as present by the user, and from the clinical history, via the relevance module 201, where the relevant information is provided. According to the above example, relevant information from the clinical history) are assigned values of {0, 1} in the diagnostic engine, which represent ‘absent’ and ‘present’.

FIG. 5 illustrates how the system of FIG. 2 can ask follow-up questions to the patient. FIG. 5 illustrates a method of medical diagnosis in accordance with an embodiment, which may be performed on the system illustrated in FIG. 2 for example. In step S119, the system of FIG. 2 makes a diagnosis (for example using the method explained above). The system then determines which further questions should be asked of the patient 101. In an embodiment, the next further questions to be asked are determined on the basis of questions that reduce the entropy of the system most effectively.

In the method illustrated in FIG. 5, the system has a pre-determined number of questions to ask the user. In S315 it is determined whether this number of questions has been reached. If not, the probabilities of the different diseases are considered and the question to be asked is determined (e.g. that which reduces the entropy of the system most).

Once the user supplies further information, then this is then passed back to the diagnosis engine 111 to update evidence to produce updated probabilities. The evidence vector is updated with the new information. Further iterations may be performed until the allowed number of questions is reached. At this point a final diagnosis is output. Where the new evidence obtained from the user conflicts with the previous evidence, this may be resolved by implementing a rule which gives priority to the information provided by the user for example. The input to the model then comprises the user information (e.g. that “headache” is present) instead of the previous information (e.g. that “headache” is absent).

In the above described example, the system has a pre-determined allowable number of questions. However, alternatively, the system may determine whether a diagnosis is accurate and then determine whether to ask further questions. In this case, the diagnosis engine 111 determines:

P(Disease_i|Evidence) for all diseases

P(Symptom_i|Evidence),

P(Risk factor_i|Evidence).

It is possible to use a value of information analysis (Vol) to determine from the above likelihoods whether asking a further question would improve the probability of diagnosis.

For example, if the initial output of the system seems that there are 9 diseases each having a 10% likelihood based on the evidence, then asking a further question will allow a more precise and useful diagnosis to be made. Further iterations may be performed until a diagnosis is obtained with sufficient certainty.

The evidence may comprise a vector, where elements of the vector corresponds to one particular item of medical data, and the value of the elements is 0 or 1 (representing absent or present). Further elements in the vector corresponding to the other nodes in the model may be assigned a value of 0.5 for example. Each piece of medical data corresponds to a concept (symptom, risk factor or disease) which is used in the model. The diagnostic engine may receive a first vector representing the user input, and a second vector representing the input from the clinical history. Elements of the first vector representing the user input may have values of ‘1’ for all symptoms, diseases or risk factors positively identified by the user as present, and values of ‘0’ for all symptoms, diseases or risk factors that are positively identified as absent. A value of 0.5 may be assigned for elements where the presence/absence is unknown. Elements of the vector representing the clinical history may have values of ‘1’ for all relevant symptoms, diseases or risk factors that are present, values of ‘-1’ for all relevant symptoms, diseases or risk factors that are validly absent, and values of ‘0.5’ for the other symptoms, diseases or risk factors.

Both vectors may be of the same length, with each element denoting a particular concept according to the above. Each entry may correspond to a concept used in the model for example. The information is combined in the manner described below. The data that is passed to the inference engine 109 and the PGM 120 of the diagnosis engine 111 is derived from both the user input in S107 and the clinical history in S119. The vectors representing the user input and the clinical history may have different values—intuitively, this is expected because, for example, the user is expected to report symptoms that he is currently experiencing, while the clinical history will provide other concepts such as risk factors, symptoms, and diseases previously reported.

Where both of the first vector and the second vector indicate that the concept is present, the concept is indicated as present (1) in the input evidence. Where one of the first vector and the second vector indicates that the concept is present, and the other indicates that it is unknown, the concept is indicated as present (1) in the input evidence. Where both of the first vector and the second vector indicate that the concept is unknown, the concept is indicated as unknown in the input evidence (0.5). Where both of the first vector and the second vector indicate that the concept is absent, or where one of the first vector indicates that the concept is absent and the other indicates that it is unknown, the concept is indicated as absent (0) in the input evidence. Where one of the first vector indicates that the concept is present, and the other indicates that it is absent, there is a conflict and this is resolved using a pre-defined priority.

In an embodiment, in order to combine the information from the clinical history and the user input, a set of rules are implemented to resolve any conflicts. The rules reflect the priority of the information source. For example, information from the clinical history that originates from a doctor is prioritised over information from the user. Information from the user is prioritised over information from the clinical history from any other source. In this manner, a single input vector may be generated. For example, if the user inputs that they do not have asthma, but the clinical history comprises information from a doctor indicating that they do have asthma, the information from the clinical history is taken and asthma is indicated as present. Additional or alternative rules may be implemented, for example prioritising more recent information. The data may be stored together and read from a system wide event bus. The resolution of conflicts is achieved based on policies.

In an alternative embodiment, a conflict may be resolved by requesting further confirmation from the user.

Conflicts refer to the case where information indicating both presence and absence of the same concept is provided. Where one of the user input or clinical history indicates unknown in relation to the concept, there is no conflict, and the information from the other source is taken. Where both the user and input and clinical history indicate the same, again there is no conflict, and the information from either is taken.

Conflicts may also occur within the clinical history information. Resolution of such conflicts is performed before the combined input evidence is generated. It may be performed at the relevance module 201 for example. Such internal resolution may be performed on the same or different pre-defined priorities. This will be described in more detail below.

The combined vector is then passed as input to the PGM for the calculation of the probabilities.

The diagnosis system described above can be used in the following manner. An unwell patient inputs symptoms such as headache and short of breath. The system then asks a set of questions to the user to determine what could be their diagnosis, as described in relation to FIG. 5. During the flow of questions, additional information about the user, for example, they have a family history of cardiac problems and they used to smoke will be received. Then, the system will generate a set of one or more diagnostics, such as the patient might have angina or other cardiovascular disease and book an appointment with a GP (General Practitioner). During the appointment, the GP will be able to see in the patient history all of the data points populated during the diagnosis, the presenting complaint and the possible diagnostics. The GP will give a diagnostic, write a prescription for some medication and write some notes about the appointment. The User Graph will process those notes to extract only the medical concepts and store them to the clinical history of the patient. All of the information recorded into the User Graph records are coded into concept mapping to the Knowledge Base (KB).

The information may need to be confirmed by the user at the time of use before it can be integrated into medical decision-making. For example, this may be required for clinical safety reasons. Therefore, providing of all historically-gathered information about a user could result in the need for their validation of an overwhelmingly long list of concepts. This would make an extremely poor user experience. It also results in a large amount of data transfer between the diagnosis system 111 and the user device 102. For this reason, it may be desired to cap the list of concepts to be confirmed. For example, a maximum of 10 concepts per flow may be confirmed. The concepts to be confirmed may be selected based on the measure of relevance. Only information about those 10 concepts are then used during the diagnosis flow however, instead of the full clinical history evidence about the user.

Furthermore, users may find it confusing to be asked to confirm concepts about their previous medical history that appear entirely unrelated to their presenting complaint. For example, they may present with a headache, but are immediately asked to confirm whether they are still pregnant, or whether they truly have a family history of liver cancer. This also makes for a poor user experience.

Providing the clinical history information about the user that is considered medically relevant to their presenting complaint identifies the most impactful subset of concepts to confirm with the user and therefore to use in the flow. Also, it is much more likely that the concepts extracted by the relevance filter will make sense to the user as concepts to be asked about, as they will appear semantically related to their query. FIG. 2(d) illustrates how the relevant clinical history may be presented to the user.

In the above described examples, the relevance module 201 is configured to generate a sub-set of relevant concepts from those returned from the clinical history. However, optionally, the relevance module 201 may perform additional functionality.

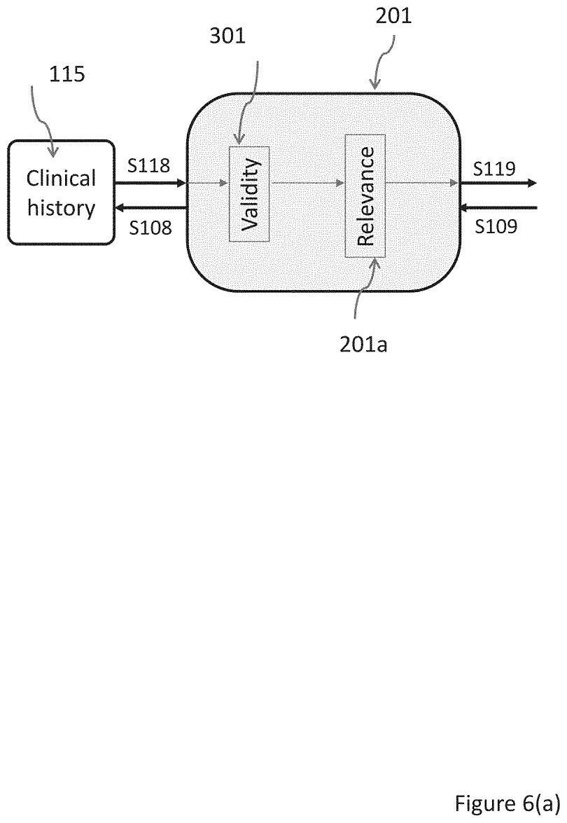

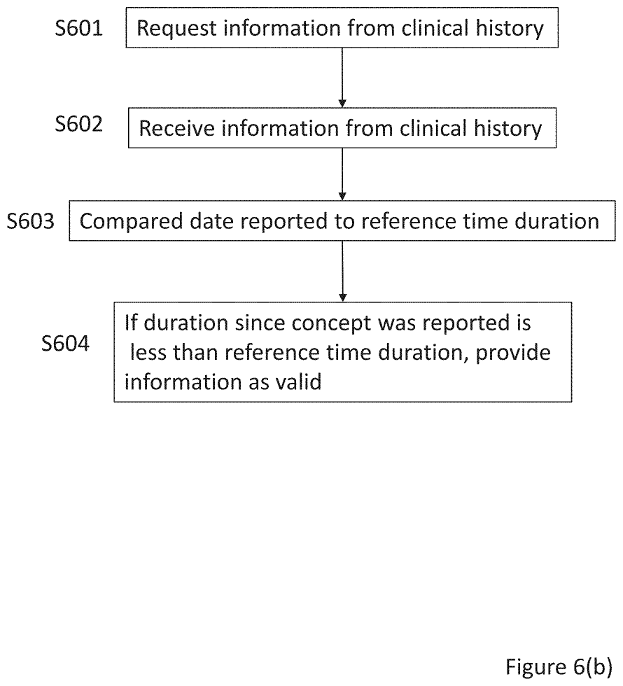

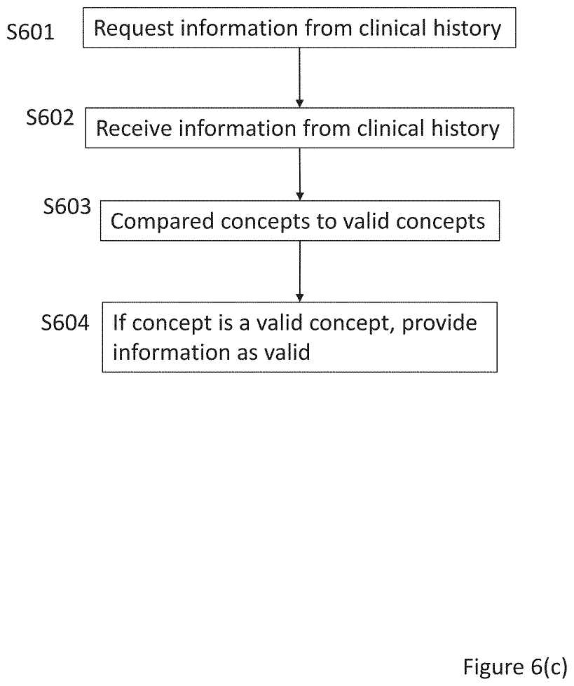

For example, prior to determining the relevance, the relevance module 201 may determine the validity of the information returned from the clinical history. This is described in relation to FIG. 6(a) below. FIG. 6(a) is a schematic illustration of a system 201 in accordance with an embodiment for determining the relevance of medical information, in which validity of the information is determined prior to relevance. The validity module 301 obtains information regarding a items of medical data associated with a user and stored in a clinical history 115. It determines the validity of the information relating to some or all of the items, e.g. symptoms, risk factors and/or diseases, and provides the valid information as output.

The relevance module 201 extracts concepts from the events in the clinical history. The relevance module 201 comprises a validity module 301. The validity module 301 assigns 1, −1, or 0 to the extracted concept to indicate whether the concept is validly present (1), validly absent (−1) or this is unknown (0), and outputs a list of concepts, each with an assigned value of 1, −1, or 0. In another example, the validity module 301 outputs a list of concepts which are identified as validly present in the clinical history. In yet another example, the validity module 301 may output a list of concepts together with a confidence level, C, where −1≤C≤1. The closer to “1” the value of C is, the higher the likelihood that a concept is validly present; and the closer to “−1” the value of C is, the higher the likelihood that a concept is validly absent.

At the diagnostic engine, C may be compared to one or more threshold values in order to generate an input to the model. If a concept has a value greater than or equal to a first threshold value, it is considered to be present. If a concept has a value less than or equal to a second threshold value, it is considered to be absent.

An example code that shows the manner in which the confidence level is used by the diagnostic engine to generate the model input is described below.

For example, the input “evidence_set” that is inputted to the model may be obtained according to the code below. A node corresponding to an item of medical data can be present, Evidence(node=node, state=PRESENT)”, or absent, “Evidence(node=node, state=ABSENT)”. In the example below, the state of the node is determined by comparing a confidence value from the validity module 301, “validity.confidence”. Since the PGM model is based on probabilities then PRESENT becomes probability 1 and ABSENT becomes probability 0. All other nodes may be assigned a probability of 0.5 for example.

evidence_set = EvidenceSet( )
evidence_set.add_all(

Evidence(node=node, state=PRESENT)
for node, validity in node_to_validity.items( )
if node.is_boolean
if validity.confidence >= PRESENT_CONFIDENCE

)
evidence_set.add_all(

Evidence(node=node, state=ABSENT)
for node, validity in node_to_validity.items( )
if node.is_boolean
if validity.confidence <= ABSENT_CONFIDENCE

)

PRESENT_CONFIDENCE is a first threshold used to determine whether the state is present. In an embodiment, PRESENT_CONFIDENCE=1. ABSENT_CONFIDENCE is a second threshold used to determine whether the state is absent. In an embodiment, ABSENT_CONFIDENCE=−1. For values of the validity.confidence which do not meet either threshold, state=0.5 may be assigned for example.

The output of the validity module 301 may comprise the information shown in the examples below. The output of the validity module 301 comprises the valid information. The first example below shows a disease “Asthma” having a confidence=−1.

{

“concept”: {

“baseConcept”: {

“label”: “Asthma”,
“iri”:“https://protect-

eu.mimecast.com/s/9nlFC1wGKIM3E0MtkryYo?domain=bbl.health”

},
“modifiers”: [ ]

},
“validity”: {

“confidence”: −1.0

}

},

Another example shown below shows a disease “Crohn's disease” having a confidence=0. In this example, a modifier with label “Chronic phase” is also defined.

{

“concept”: {

“baseConcept”: {

“label”: “Crohn's disease”,
“iri”:“https://protect-

eu.mimecast.com/s/OS6rCpYkxSnmxJnSO2CbB?domain=bbl.health”

},
“modifiers”: [

{

“type”: {

“label”: “HAS QUALIFIER”,
“iri”:“https://protect-

eu.mimecast.com/s/Xe1oCqxlyu8Z7x8F0oDpX?domain=bbl.health”

},
“value”: {

“label”: “Chronic phase”,
“iri”:“https://protect-

eu.mimecast.com/s/X5NjCrkmzT8EDz8FMx_BZ?domain=bbl.health”

}

}

]

},
“validity”: {

“confidence”: 0.0

}

}

A further example below shows a disease “Family history of malignant neoplasm of thyroid” having a confidence=1.

{

“concept”: {

“label”: “Family history of malignant neoplasm of thyroid”,
“iri”:“https://protect-

eu.mimecast.com/s/A4KBC31yMipqRPpsg80AsX?domain=bbl.health”

},
“validity”: {

“confidence”: 1.0

}

}

In the above examples, “confidence”: −1.0 represents validly “absent”; “confidence”: 0 represents “unknown”; and “confidence”: 1.0 represents validly “present”. Furthermore, in the above examples, the concepts are items of medical data, e.g. Asthma is an item of medical data, while the information about the medical data is the confidence, i.e. whether the concept is validly present, validly absent, or this is unknown. The values of −1.0, 0, and 1.0 are assigned to the concepts by the validity module 301. Example methods of assigning the confidence will be described in more detail below.

The validity module 301 takes as input the information from the clinical history 115 of the user, as described above, and extracts a set of symptoms, risk factors and/or diseases to be used in the probabilistic model. The validity module 301 determines the information corresponding to each of the set of symptoms, risk factors and/or diseases to be used in the probabilistic model from the stored information associated with the user (for example whether the symptoms, risk factors and/or diseases are validly present, validly absent or this is unknown in this clinical history).

For example, the clinical history 115 of the user may comprise information from a doctor's note indicating that the patient has diabetes. The information about symptoms, risk factors and/or diseases from the clinical history 115 of the user therefore comprises an indication that the disease “diabetes” is present. If this information is determined to be valid, the validity module may generate an entry “1” corresponding to the IRI representing the concept “diabetes”. The absence of a symptom, risk factor and/or disease may be indicated by an entry of “−1” for example. If it is unknown whether the symptom, risk factor and/or disease is present or absent, this may be indicated by a 0. As previously stated, other means of indicating this information may be used however.

By comparing the symptoms, risk factors and/diseases from the diagnostic engine to the information about the symptoms, risk factors and/or diseases from the clinical history, information about the symptoms, risk factors and/or diseases used in the model is obtained (for example, whether each is validly present, or validly absent or this is unknown).

Thus the validity module 301 first retrieves information relating to items of medical data from the stored clinical history 115, and then determines the validity of the information for each item of medical data corresponding to the set of symptoms, risk factors and/or diseases to be used in the probabilistic model. This may comprise determining which of the symptoms, risk factors and/or diseases which are indicated as being present or absent in the clinical history 115 are validly indicated as such. Various methods of determining the validity are described below in relation to FIGS. 6(b) and (c). The validity module 301 then passes valid information relating to the symptoms, risk factors and/or diseases to the relevance stage 201a. The relevance stage 201a determines the relevance of each concept returned from the validity module as being present or absent to the patient input, and outputs the relevant information, in the same manner as has been described previously.

In a medical diagnosis system such as described in relation to FIG. 2, the relevant, valid information is passed to the diagnostic engine 111. This information is taken as input to the probabilistic model 112 (as well as the information from the user input). The probabilistic model 112 determines the probability of the user having each of one or more diseases from the input information, as has been described previously.

Various methods by which the validity module 301 determines if the information relating to a symptom, risk factor and/or disease is valid in accordance with embodiments will be described in more detail below in relation to FIGS. 6(a) and (b). The validity module 301 may output the set of IRIs representing the symptoms, risk factors and/or diseases used in the probabilistic model, together with the valid information (for example, the information may indicate, for each concept in the model, whether the concept is validly present, validly absent or whether its presence/absence is unknown).

The validity module 301 receives information from the clinical history, comprising one or more concepts and information indicating that the concept is present or absent. The validity module 301 determines, for each concept corresponding to a symptom, risk factor and/or disease used in the probabilistic model, whether the concept is validly present, validly absent or whether its presence/absence is unknown. For example, if the validity module 301 determines from information in the clinical history that a symptom is present, the validity module 301 then determines whether this is valid. If this information is valid, the validity module 301 outputs that the symptom is present (1). If there is no information in the clinical history regarding the presence or absence of the symptom, or the information in the clinical history is determined to be not valid, the validity module 301 outputs that the presence/absence of the symptom is unknown (0). Similarly, if the clinical history provides information from which the validity module 301 determines that a symptom is absent, and this information is determined to be valid, the validity module 301 outputs that the symptom is absent (−1).

Data about the patient is held in the patient's clinical history 115. The clinical history comprises a record of information relating to items of medical data (i.e., symptoms, diseases, and/or risk factors) previously reported by the diagnosis engine 111. The clinical history may also include information relating to items of medical data from notes by a human doctor, prescriptions, user reported symptoms or events, healthchecks, lab tests, healthcare providers' databases or medical data from other sources. In an embodiment, information indicating the source of each entry is also included with each entry. For example, information identifying whether the entry originated from a doctor, user, medical diagnosis system, or another source may be included.

The clinical history may contain entries over a large range of time e.g. hours, days, weeks, months, or years. In an embodiment, the clinical history further comprises temporal information, corresponding to the reporting time of each entry. For example, each entry in the clinical history may comprise date information, indicating the day on which the entry was reported. For example, an entry in the clinical history may comprise an IRI representing a symptom, disease or risk factor entered by a doctor at an appointment (for example “backache”) and the date of the appointment. An entry previously reported by the diagnostic engine 111 may comprise a symptom input by the user together with the date on which the input occurred, and a second entry may comprise the disease determined by the diagnostic engine, together with the date on which the diagnosis occurred. Although date information is given here as an example, the temporal information may alternatively comprise time information, or only the year for example. Date information may be stored so that the validity module 301 may determine validity based on the time since the concept was reported. However, validity may be determined using alternative criteria, and therefore in some examples the temporal information is not stored.

Items of medical data in the clinical history may comprise concepts, in particular the IRI(s) of the concept. The clinical history may also store information indicating whether the concept is present, absent or unknown. For example, unary concepts (e.g. NOT headache) may be used to indicate that a concept is absent. Furthermore, the clinical history may include temporal information about concepts—that is, when were they were reported. It may further comprise information about the source of the information (for example doctor, patient, diagnosis system). The clinical history may store, for a particular patient: the Patient Id, event information comprising one or more concepts and information indicating whether each concept is present or absent, the date for each event, source for each event, and any extra application specific info (e.g. the confidence of a diagnosis or the free text the concepts were derived from).

Each entry in the clinical history thus may further comprise temporal information, for example date information indicating the day on which the entry was reported. It may additionally or alternatively comprise information about the source of the information (for example doctor, patient, diagnosis system or other).

FIG. 6(b) shows a flow chart illustrating a method of determining validity used in a method in accordance with an embodiment. In step S108 the relevance module 201 requests information from the patient's clinical history 115. This corresponds to S601 in FIG. 6(b). The clinical history service provides information to the validity module 301 in step S118. This corresponds to S602 in FIG. 6(b). The information provided to the validity module 301 may comprise concepts. The information may indicate whether the concept is present (for example by including the concept) or absent (for example by including the operator “NOT” together with the concept). Alternatively, the information may simply indicate that the concept is present. For each entry, the information may further comprise temporal information (when were they were reported). It may further comprise information about the source of the information (for example doctor, patient, diagnosis system).

In S603, the validity module determines the validity of the information. In an embodiment, the validity module 301 is configured to determine whether information relating to a concept (for example presence or absence of items of medical data such as symptoms, diseases and/or risk factors) recorded in the past is still valid at present. The validity is determined based on the temporal information. The date on which the concept was reported is compared to a stored reference time duration in this example.

For example, suppose an event of a headache has been reported 3.3 months ago. It is unlikely that such an event is relevant to a diagnosis in the present, and therefore such information is determined not to be valid (i.e. the presence of a headache is determined to be invalid and, in the absence of any other information relating to a headache, whether a headache is present or absent is reported as unknown). In another example, suppose an event of a chest pain has been reported 3 days ago. It is likely that such an event is relevant to a diagnosis in the present, and therefore such information is determined to be valid (i.e. the presence of chest pain is determined to be valid). In yet another example, suppose a condition of ‘Diabetes’ is associated to a patient. It is almost certain that such an event is relevant to a diagnosis in the present, and therefore such information should be tagged as valid.

Temporal information relating to when the concept was reported is compared with information indicating how long information relating to the particular concept is valid, in order to determine whether the specific information is valid at the current time.

In this example, the validity module 301 comprises a stored list of items of medical data, encoded as concepts, and the relevant time durations for which they remain valid. The validity module 301 determines the time since the entry was reported by comparing the current date and the date on which the information was reported.

The validity module 301 compares the time since the entry is reported with stored information relating to each concept, indicating the duration of time for which information relating to that concept is valid. The stored information indicating the duration of the validity may be generated by human doctors for example. If the time since the entry is reported is longer than the time for which information relating to that concept is valid, the information in the entry is determined to be invalid. The validity module 301 outputs “unknown” in relation to the concept (indicated by a 0). If the time since the entry is shorter than the time for which information relating to that concept is valid, the information in the entry is determined to be valid. The validity module 301 outputs the valid information in relation to the concept (for example present, indicated by 1 or absent indicated by −1).

The output from the validity module 301 is provided to relevance stage 201a. Thus in S604, the valid information is provided.

The validity of an input concept is determined based on the time when it was reported. The time between the concept being reported and the validity of the concept being determined is termed a “time duration”. A reference time duration is a time duration for which a concept is known to be valid. It may be a time duration for which a concept is known to be valid within a certain confidence interval (e.g. 90%) for example. The reference time duration may be generated by a human expert for example. The method comprises: looking up reference time durations for which information about symptoms, diseases and/or risk factors are valid from a table, for each symptom, disease or risk factor, comparing the reference time durations to the time duration from when the symptom, disease or risk factor was reported, and providing information relating to the symptom, disease or risk factor obtained from the clinical history if the reference time duration is greater than or equal to the time duration from when information about the symptom, disease or risk factor was reported.

The validity module thus comprises a database that contains a list of concepts and an associated reference duration, which indicates for how long a concept remains valid. The reference duration may indicate how long a concept remains valid within a certain level of certainty e.g. 90%, after an incidence of the concept has been reported. The information in this database may be entered by human experts.

Although in the above example, whether the information is valid is determined in the same manner regardless of whether the information indicates that the concept is present or absent, in an embodiment, the validity may be determined differently for each case. Thus it may first be determined whether the information indicates that the concept is present or absent. If present, a first criteria is applied in order to determine validity. If absent, a second criteria is applied. This is because the two cases may not be symmetrical, especially in the case of symptoms. This may mean using a shorter validity duration for absent information for example. The duration for absence may be based on statistics on how likely a patient is to acquire the condition for example. It may mean confirming the absence with the user.

The examples illustrate simple concepts such as headache, however, the validity module 301 may also operate on complex concepts (e.g. Severe Headache).

In the above described example, the validity module 301 matches the received concepts from the clinical history with the stored reference duration associated with the concept.

In an embodiment, the validity module 301 is configured to predict the duration for which a concept remains valid based on the duration of a related concept, the related concept being either parent or a child.

However, FIG. 6(c) illustrates an alternative method in which a table storing a set of concepts for which the information indicating that the concept is present is permanently valid is stored. For example, information indicating the presence of diabetes is likely to be relevant to a diagnosis forever. In this example, only information indicating the presence of a concept is returned from the validity module. FIG. 6(c) shows a flow chart of a method of determining validity in accordance with an embodiment. In step S601 the relevance module 201 requests information from the patient's clinical history 115. In this example, the clinical history may provide information including concepts which are present in the clinical history in S602. Temporal information is not provided. The validity module 301 compares the list of concepts from the clinical history with the list of valid concepts in S603 to determine validity. Only information relating to the valid concepts is output in S604.

Concepts for indicating permanence may also be stored in the knowledge base, and used by the validity module to determine which concepts are permanently valid. For example, permanence may be represented by the simple concept “Is Permanent”. One or more concepts used in the model may comprise a simple concept indicating permanence. The validity module may use this information to determine whether information provided from the clinical history is valid, instead of a stored table.

Conflicting data points may exist in the clinical history. In an embodiment, the relevance module 201 reconciles differences between entries in the clinical history. For example, the clinical history may provide two entries, relating to the same concept, to the relevance module 201. One may indicate that the concept is present and the other may indicate that the concept is absent. In this case, there is a conflict within the clinical history.

In an embodiment, a set of rules are implemented to resolve such conflicts. These may be applied before relevance is determined, for example they may be applied before the valid clinical history is transferred to the relevance stage 201a in the system of FIG. 6(a). In an embodiment, the rules reflect the priority of the information source. For example, information that originates from a doctor is prioritised over information from the patient or from the medical diagnosis system. For example, if one entry in the clinical history originates from the user and indicates that they do not have asthma, and another entry in the clinical history comprises information from a doctor indicating that they do have asthma, the information from the doctor is taken and asthma is indicated as present.

Where one entry indicates unknown, the information from the other source is taken since no conflict exists. Where both sources indicate the same, the information from either is taken as again, no conflict exists.

Other rules may additionally or alternatively be implemented, for example a more recent entry may be taken as having priority. Different policies may be specified, for example doctors may have priority over self-reports, or it can be specified to trust one particular source only.

As explained above, the clinical history comprises a record of items of medical data (i.e., symptoms, diseases, or risk factors) previously reported by the diagnosis engine 111. The clinical history may also include notes from a human doctor, prescriptions, user reported symptoms or events, or medical data from other sources. However, information recorded by the user over time may contradict the risk factors or diseases flagged by the human doctor. If conflicting concepts are passed to the diagnosis engine, the diagnosis may not be accurate. In this case, where valid information about the same concepts are conflicting, the validity module 301 may be configured to pass information that has been entered by a human doctor, rather than that logged in the clinical history by the user for example.

In the above described examples, stored embeddings corresponding to concepts in the clinical history and in the patient input are retrieved, in order to determine a similarity value, used as a measure of relevance. The embeddings are generated during a training stage, and stored for use during implementation. In the below, various example methods of generating the embeddings are described.

Relevance may be considered in the following manner: a concept is relevant to an input concept if it increases the accuracy of diagnosis of the disease from which the input concept results. In other words, whether or not something is relevant depends on whether it helps to achieve the specific goal of diagnosis.

Similarity can be considered a sub-class of relevance. The term “similar” generally means that something shares common characteristics with another concept. It should be noted that the term similarity used here is not the same as a similarity measure, such as the cosine similarity, which is a mathematical operation on two vectors.

Taking the example of a clinician during a consultation: their aim is to use the information about the patient that are available to them to come to the correct diagnosis of that patient's condition. To help them do this, they will often ask the patient questions or take quantifiable measurements (e.g. from blood test results) to both validate suspicions (shown in their patient history/reported by the patient) and fill in gaps in their knowledge about that patient. This information gathering and validation process must be balanced with efficiency—which is why clinicians only ask questions or take measurements about aspects of the patient which they believe will help them decide on the correct diagnosis in the current scenario as quickly as possible. All such information used in their decision-making—both those that represent positive signals and those that represent negative signals—are therefore considered relevant to the goal. The information they seek to gather will give them the clearest sense of direction, either through positive signals (affirming the test hypothesis) or through negative signals (discounting “competitor” hypotheses).

For example, a patient presents with a complaint of dizziness. Their patient history shows that they have recently complained about the following: a) Benign Positional Vertigo, b) Fast Heart Rhythm, and c) Tendonitis in Left Wrist. The Clinician must decide which of these concepts to validate with the patient. a) Benign Positional Vertigo is Similar because Vertigo and Dizziness are different ways to describe the same symptom. Benign Positional Vertigo is Relevant to the current case (dizziness) because it is a condition that could explain dizziness symptoms. b) Fast Heart Rate is not Similar to Dizziness, but it is Relevant because the Clinician knows that Dizziness could be a sign of the condition Ventricular Tachycardia when the Patient simultaneously presents with Fast Heart Rate. c) Tendonitis in Left Wrist is not Similar to Dizziness, and also not Relevant because the Clinician knows that Tendonitis is a chronic condition for which there are no known links with dizziness nor the conditions that could cause dizziness. Therefore, the Clinician will validate concept A because it is both Similar and Relevant. The Clinician will also validate concept B because it is Relevant despite being Dissimilar. The Clinician will not validate concept C because it is Irrelevant.

From this example, it can be seen that similarity is a sub-class of relevance. Similarity can enable relevance, but concepts that are considered relevant should not be limited to those that are similar to the concept in question. Again, the term similarity is not used here to refer to the cosine similarity measure, which may be used as a measure of relevance when operating on embeddings which are trained to capture relevance.

The methods described below to generate the embeddings use measures of semantic relevance. Semantic similarity between two concepts expresses the taxonomic similarity between terms. For example, chronic bronchitis and asthma are similar because they are both respiratory disorder. On the other hand, semantic relevance is a much broader concept, that covers not only the taxonomic relationship, but may also included metonymy, functionality, cause-effect and/or others. For example, a condition and the treatment are related although they are not similar.

The methods use statistics to create representations of the concepts taken from the text to build semantic models of context. The goal is to express a measure of how much information a concept can provide to a diagnosis, given the user input. This can then be used to rank the concepts for example.

Distributed representations based methods generate a representation of the concepts in a vectorial space based on a domain corpus. This captures more of the relevance, or relatedness, rather than the similarity between concepts, as the vectors are based on the context in which concepts are used within a domain specific corpus that capture relevance rather than similarity. Therefore, the method builds a representation of the meaning of the concept based on the context they are used in. Context vector measures of semantic relevance are based on the assumption that concepts that appear in similar contexts are related. The method creates embeddings, which are vectorial representations of concepts in a low dimensional (i.e. n dimensional) space meant to represent the semantic meaning of the concept: concepts that are close together in this vectorial space are meant to be close in meaning. To create this vectorial space, the architectures such as those used for Word2Vec may be used, as is described below. Alternatively, architectures such as those used for fasttext may be used.

An example model using a word2vec type architecture comprises a single hidden layer network trained to give the probability of each concept to be in the neighbourhood (given a window size, which will be described later) of a given concept. The output probabilities relate to how likely it is to find each concept of the vocabulary near the input concept. For example, if you give the network the concept “fever”, the output probabilities are going to be higher for “headache”, than for unrelated concepts such as “diabetes”. The network learns the probabilities from the frequency with which each pair is represented in the corpus.

Using word embeddings type models provides means to vectorize the concepts. In order to be able to compare concepts, vector representations are generated. To generate the embeddings, a word embedding type model can be trained on Clinical Notes for example, with some pre-processing performed. Once the training data is pre-processed, the next step is to train and test the model.

FIG. 7(a) is a flow chart of a method of training a medical diagnosis system, comprising generating vectors corresponding to medical concepts, in accordance with an embodiment. In this example method, a word2vec type technique is used, in which a model is trained on data extracted from an electronic health record (EHR) database.

Other data may alternatively or additionally be used for training. For example, data extracted from the user graph may be used as the training data. Such data may include PGM flows, Clinical Portal consultations (including codes entered by GPs during a consultation, which are of higher quality, and codes extracted from consultation text automatically, which are generally of lower quality) and Check Base flows. Other data that may be used for training includes Check Base flows, or doctor interaction with GP portal as a feedback signal.

The method results in an embedding i.e. a vector, for each IRI in the training data vocabulary. Two embeddings will be similar to each other if the respective concepts are relevant to each other. Using word embeddings based models allows to vectorize the concepts. It is then possible to measure their relevance by computing their cosine similarity for example.

In step S701, data pre-processing is performed. For example, patient history data is extracted from the EHR database for a plurality of patients. A patient history is defined by all the medcodes (events, e.g. “asthma”) belonging to a given COMBID (unique for patient-practice combinations). The entries for each patient are ordered by date and time. The medcodes are grouped by consultation, where each consultation may be considered as a sentence in the word2vec framework, i.e. a list of concepts. The consultations are also grouped by user. A user would correspond to a “document” in the word2vec framework, i.e. a list of consultations. A medcode is a clinical code used to represent clinical terms, for example NHS read codes. All the medcode entries for each patient are then replaced with their corresponding IRI using a conversion algorithm, for example using a look up table. Any entry that cannot be converted is discarded.

Since the database might not cover many of the symptoms and risk factors in the knowledge base, each “sentence”, i.e. consultations, may be extended by including neighbouring concepts from the knowledge base. These are simply added into the list of concepts for the consultation. In order to extend the dataset and the vocabulary, some of the sequences may be duplicated and one or more concepts replaced with an ancestor concept (e.g. parent concept) from the Knowledge Base in the duplicate sequence.

The data set at this stage comprises a sequence of concepts (represented by as for example) for each consultation, and a sequence of consultations for each patient. Each sequence of concepts can be considered to be a set of concepts that are relevant to each other. Each sequence can be considered to correspond to a sentence for the purposes of applying the word2vec type training algorithm. The training data comprises sequences of medical conditions that are found in the same patient history and in the same consultation.

The resulting set of concept sequences makes up the training data for the model. Various publically available training algorithms can be used to train a word2vec based model using a set of training data. Details of the word2vec technique are provided in “Distributed Representations of Words and Phrases and their Compositionality”, Mikolov et al, arXiv:1310.4546, 16 Oct. 2013, the contents of which are hereby incorporated by reference.

Different word2vec type models may be used, including a skip-gram type model or a Continuous Bag or Words (CBOW) type model. Although examples based on word2vec algorithms are described below, alternative algorithms, such as the fasttext algorithm may be used. Although the term “word2vec” type model is used, the model is used to generate embeddings for concepts rather than words, as will be described below.

A schematic illustration of an example word2vec type model is shown in FIG. 7(b). The model comprises a two-layer neural network that processes input concepts. The input to the model is a vector of length V, where V is the number of concepts in the training data. The input vector represents an input concept from a training sequence, i.e. consultation, represented using one-hot encoding. The entry in the vector corresponding to the input concept from the sequence is given a value of 1, with the other entries having a value of 0. For a sequence in the training data, a sliding window may be used to extract the target and context concepts. For the case where the first concept in a sequence from the training data is “headache”, the first input vector comprises a 1 in the position corresponding to the concept “headache”, and a 0 in all other positions, as shown in FIG. 7(b).

The model comprises a hidden layer, comprising n linear nodes. The weights between the input layer and the hidden layer correspond to the vector encodings for all of the words in the vocabulary. These weights are learned during training, and stored as the vector embeddings corresponding to each concept. The parameter n corresponds to the length of the embeddings. In an embodiment, n is 128. No activation function is used in the hidden layer. Furthermore, no bias terms are included.

For each entry, i, in the input vector of length V, the set of weights which multiply the entry i make up an n dimensional vector which is stored as the embedding for the concept i. Thus there are V embeddings, each of length n, which are stored.

The output layer also comprises V nodes. Each node again corresponds to one of the words in the vocabulary. The softmax function is applied to the output layer. Each output node also has a corresponding set of weights, which is multiplied by the output of the hidden layer, and the softmax function applied to the result. The softmax function takes as input a vector of real numbers, and normalizes it into a probability distribution.

Each output node gives the probability corresponding to a concept. There is one output node for each concept in the vocabulary. The output node gives the probability that a concept at a randomly chosen nearby position in the training sequence is the concept corresponding to the node.

The input is thus a V-dimensional vector, with V the number of concepts in the corpus. The hidden layer has n neurons, with n being a hyperparameter of the model. The model comprises a single hidden layer, and is a fully connected neural network. The neurons in the hidden layer are all linear neurons. The input to hidden layer connections can be represented by matrix of size V×n, with each row being the embedding for a vocabulary word. In same way, the connections from hidden layer to output layer can be described by matrix of size n×V.

The model takes as input a one-hot vector representing the input concept and the training output is a one-hot vector representing the output concept of the neighbouring concept.

The model output is a vector of probability distributions. The weights of the hidden layer represent the projection of each of the concepts in the corpus into the low-dimensional (n dimensional) space.

The model outputs a vector of probabilities. The first entry in the output vector corresponds to “Migraine” for example. The output probability for this entry is the probability that the concept “Migraine” is at a randomly chosen nearby position to the concept “headache” in the training sequence.

In order to learn the weights, backpropagation is performed to compute the gradient of a loss function with respect to each weight in the model. Gradient descent is then performed to update the weights via optimization. Each input concept is taken in turn, (in the form of the one-hot vector of length V) and the corresponding context concept (e.g. the next concept in the sequence) from the training example may be used to generate the loss. Training is thus performed by feeding in the input concept from the training example, producing a vector of length V as output. The output may be used to determine a loss. The gradient of the loss with respect to each of the weights is determined through back-propagation. The gradients may then be used to determine the updated weights, using a gradient descent based optimiser function.

The above describes a word2vec based model which learns on pairs of words from the sequence. However, a continuous bag of words (CBOW) based word2vec type model may be used, in which multiple input concepts are used for a given output concept. In this case, the model is modified by replicating the input to hidden layer connections C times, and adding a divide by C operation in the hidden layer neurons. C is the number of context concepts in the window, and is a hyperparameter. In an embodiment, C is equal to 5. Each of the C input concepts is encoded using a one hot vector of length V, in the same manner as described above. The output of the hidden layer is the average of the vectors corresponding to the input concepts. The set of input concepts is generated by a sliding window through the set of concepts in a consultation. The input weights are tied for the C input concepts, and are stored as the concept embeddings.

Alternatively, a skip-gram type model may be used. In this case, multiple output concepts are used for a given input concept. The output layer is replicated C times. C output vectors are thus produced, and the error vectors from all output layers are summed during backpropagation. Again, C is selected as a hyperparameter, In an embodiment, C is equal to 5. The input weights are stored as the concept embeddings.

As has been explained previously, the number of dimensions for the embeddings n, corresponding to the number of nodes in the hidden layer, can also be selected as a hyper-parameter. In an embodiment, there are 128 entries in the embeddings. Alternatively however, a length of 32 or 512 could be used for example.

Other hyper-parameters may be selected. For example, the epoch number (the number of times the training vectors are used to update the weights) may be selected. In an embodiment, 5 epochs is used.

In an embodiment, a CBOW type word2vec algorithm is used, with window size 5, for 5 epochs, and with 128 dimensions for the embeddings.

Once the model is trained, the resulting embedding vectors (i.e. the weights) are stored, and may then be compared using cosine similarity as has been described above in order to determine the relevance of a concept from the clinical history to a concept in the patient input.

An input training sequence may comprise the concepts {“headache”, “migraine”, “fever”, “dry mouth”, . . . }. A first training example is extracted from this sequence in S702. The input concept (or concepts in the CBOW case) is converted into a one-hot vector comprising a 1 in the position corresponding to the concept “headache”, and a 0 in all other positions and taken as input to the model in S703.

In order to learn the weights, backpropagation is performed to compute the gradient of the loss function with respect to each weight in the model in S704, and gradient descent is then performed to update the weights via optimization. Each input is taken in turn, and the corresponding concept (or concepts in the skip-gram case) from the training example used to generate the loss function. The weights may be updated in batches comprising multiple sequences for example.

Once the training stage is complete, the weights between the input layer and hidden layer are extracted, and stored as the concept embeddings in S705. In this way, the algorithm can learn the meaning of the medical conditions based on the other conditions that are reported by a patient.

FIG. 8 shows an alternative method of generating the concept embeddings, which is used in a method in accordance with an embodiment. In addition to the method described in relation to FIG. 7, the links between concepts that are stored in the knowledge base are utilised. This allows embeddings to be generated for concepts which are not present in the training data, but which are present in the knowledge base, for example.

In this method, embeddings may be generated for concepts which are present in the knowledge base but which are not present in the training data set. For example, around 35,000 concepts may be included in training data set, whereas the knowledge base may comprise more than 1.6 million concepts. In this method, an embedding may be generated for nodes in the knowledge base, using the neighbours in the knowledge graph, as will be explained below. Concepts that are missed by the word embedding model (for example if doctors never mention a condition but always mention its parent or children in the context of consultations) may therefore be included. By combining a word embedding based approach such as described in relation to FIG. 7, with information from the knowledge base, coverage of concepts not in the training data-set may be achieved.

The method uses the structure of the knowledge base. Knowledge-based similarity measures are used to find concepts which are similar to a target concept. An embedding for the target concept can then be generated from the existing embeddings of the similar concepts. Similar concepts in the knowledge base may be found in various ways. For example, concepts which are close in the ontology are more likely to have a close semantic meaning. The degree of information carried by a link depends also on its position, since the lower the concept is in the ontology hierarchy, the higher its specificity.

Furthermore, links between general concepts convey less similarity than the ones between specific concepts.

The below method uses an information content based similarity measure, which measures the degree of specificity of a concept, to determine the similar concepts.

Information content is a knowledge based similarity measure. Other knowledge based similarity measures may be used however. Information content was found to give good results for medical ontologies.

In the method of FIG. 8(a), steps S801 to S805 correspond to steps S701 to S705 described previously. At this stage, a number of embeddings have been generated and stored for each concept in the training data set, as has been described previously in relation to FIG. 7. Description of these steps will not be repeated.

In the subsequent steps S806 to S811, for a target node in the knowledge graph, the similarity of one or more of its neighbours is calculated. An information content-based similarity measure may be used. The neighbours having a similarity measure higher than a pre-determined similarity threshold are then determined. This is extended to their neighbours, and so on. The mean embedding of all the similar concepts that have an existing embedding, for example generated in steps S801 to S805, is then calculated. This is assigned as the embedding of the target node.

In S806, a target node in the knowledge graph is selected. The target node may be a node without an existing embedding for example. Alternatively, the process may be performed in order to update existing embeddings. The direct hyponyms and direct hypernyms of this node (i.e. the direct parent and child nodes) are then retrieved. These are referred to as the “neighbour” nodes, i.e. the direct parent and child nodes of the target node in the knowledge graph.

The knowledge graph is a directed acyclic graph (DAG), comprising a finite number of edges and nodes, with each edge directed from one node to another. The graph is acyclic, which means that there is no way to follow a consistently-directed sequence of edges that eventually loops back to a same node.

In an embodiment, information about all of the nodes is generated once and then cached. This means that the hyponyms and hypernyms do not have to be determined repeatedly, but are simply retrieved for a particular node when needed. A process of generating this information for all of the nodes is therefore described here.

The nodes linked by a “subClassOf” edge in the knowledge base are extracted. The edges are also extracted into a csv file for example, i.e. a list of the relations. The number of nodes extracted in the example knowledge graph is 1,640,384 and the number of edges is 1,048,576. This process may comprise looping over all the edges, and caching the direct hypernyms and hyponyms in hashmaps. A hyponym is a word or phrase whose semantic field is more specific than its hypernym. For each edge [node 1→node 2], node 2 is saved as a hyponyms of node 1, and node 1 is saved as a hypernym of node 2. All the direct hypernyms and direct hyponyms of a specific node are retrieved. All of the ancestors and all the descendants of each node are then found and cached into a hashmap. For each node, the number of hyponym nodes without hyponyms (also referred to as “leaves”) is also computed. These can be considered as “final nodes”. Its number of ancestors is also cached.

The root of the graph is then found. The graph has only one root, which is retrieved by finding the only node with hyponyms but without hypernyms. It may be checked at this point that there is only one root. The depth of each node is also then cached. The depth is defined as the number of steps from the node to the root node. If there are multiple paths, the length of the longest path is taken. In a DAG, computing the depth may be difficult because there can be multiple ways to go from the root to a node, therefore the nodes can have multiple depth. In order to determine the real least common subsumer node (LCS, described below), the depth value is taken as the length of the longest path from the node to the root. Pre-computing the depth of all the nodes offline allows the algorithm to be a recursive one, saving computation time when looking for the LCS for a particular node.

Information about the nodes is thus cached. For each node, the information may comprise one or more of: the direct hypernyms and hyponyms, a complete set of ancestors and descendants, number of ancestors, number of leaves, information content and depth. By determining this information once, the individual computations are speeded up. For example, this information may be saved when building the graph.

The similarity to the target node of each of the direct neighbour nodes retrieved in S806 is then calculated in S807. This may be calculated by computing an information content based similarity measure. Information Content (IC) based similarity measures estimate the specificity of a concept. Various information content coefficients may be used. An intrinsic Information Content coefficient is based on the structure of the taxonomy rather than the statistic of a corpus for example. Concepts found very high in the taxonomy are likely to be non-specialized and have a lot of leaves: they have low information content. On the other hand, concepts found very deep in the taxonomy with a lot of subsumers are very specialized and have high information content. Nodes having a similar information content value are likely to be similar.

Nodes on the same branch, or in the same part of the knowledge graph are also likely to be similar. Nodes for which the least common subsumer has a higher information content are likely to be in the same branch, or the same part of the knowledge graph, since having a higher information content means the least common subsumer will be lower in the knowledge graph.

Thus two nodes having a closer information content, and for which the least common subsume has a higher information content, are likely to be more similar. Intrinsic information content coefficients are based on the structure of the knowledge graph, and thus are not reliant on a specialised corpus of data.

In an embodiment, a measure of intrinsic IC based on the ratio between the number of leaves of a concept and its number of subsumers is used:

$$IC ( c ) = - log ( \frac{\frac{\vert leaves ( c ) \vert}{\vert subsumers ( c ) \vert}}{max leaves + 1} )$$

where leaves(c) is the number of leaves (concepts at the end of the branch) found in the knowledge graph under node c, subsumers(c) is the number of concepts found subsuming c (i.e. the number of ancestors) and max leaves is the total number of leaves in the knowledge graph. This measure takes into account the total number of leaves and not only the hyponyms, making the measure less dependent on the taxonomy design.

Adding the number of subsumers to the equation allows to differentiate concepts with the same number of leaves but different specificity.

The information content is computed for each node, using the cached information about each node. This cached information may be saved to a file on a disk for example.

In step S807, the similarity between two nodes is determined. To compute the similarity between two nodes, various similarity algorithms may be used. Some examples of similarity measures that may be used are shown in the Table 1 below. One of these may be used to determine the similarity. In an embodiment, a Jaccard similarity measure is used. Each similarity measure shown below returns a value greater than or equal to 0, and less than or equal to 1, where a value closer to 1 indicates the nodes are more similar.

TABLE 1
Example algorithms for determining similarity

| Similarity measure | Formula |
|---|---|
| Jaccard | $\frac{IC ( LCS ( {c}_{1} {,}_{2} ) )}{IC ( {c}_{1} ) + IC ( {c}_{2} ) - IC ( LCS {(}_{1} , {c}_{2} ) )}$ |
| Dice | $\frac{2 \cdot IC ( LCS ( {c}_{1} , {c}_{2} ) )}{IC ( {c}_{1} ) + IC ( {c}_{2} )}$ |
| Ochiaï | $\frac{IC ( LCS ( {c}_{1} , {c}_{2} )}{\sqrt{IC ( {c}_{1} ) \cdot IC ( {c}_{2} )}}$ |
| Simpson | $\frac{IC ( LCS ( {c}_{1} , {c}_{2} )}{Min ( IC ( {c}_{1} ) , IC ( {c}_{2} ) )}$ |
| Braun-Blanquet | $\frac{IC ( LCS ( {c}_{1} , {c}_{2} ) )}{Max ( IC ( {c}_{1} ) , IC ( {c}_{2} )}$ |
| Sakal and Sneath | $\frac{IC ( LCS ( {c}_{1} , {c}_{2} ) )}{2 \cdot ( IC ( {c}_{1} ) + IC ( {c}_{2} ) - 3 \cdot IC ( LCS ( {c}_{1} , {c}_{2} ) )}$ |

Each of the above example similarity measures uses the information content (IC) of the first node (target concept node c₁), the information content of the second node (neighbour concept node c₂), as well as the information content of the least common subsumer (LCS) node. The LCS node is the LCS of the first node c₁ and the second node c₂. The LCS of two nodes c₁ and c₂ is defined as “an ancestor of both c₁ and c₂ that has no descendant which is an ancestor of c₁ and c₂”. An algorithm to find the LCS of two nodes comprises the following steps:
Compute the intersection of the two set of ancestors and the two nodes (using the cached list of ancestors for each node);
Take the node with the largest depth as the LCS.

In S807, a similarity measure, being a value greater than or equal to 0 and less than or equal to 1 is generated between the target node and each of the direct neighbour nodes (the direct parent and direct child nodes of the target node), as described above. In S808, it is determined, for each of these similarity measures, whether the similarity measure meets a pre-defined criterion. For example, it is determine whether the similarity is greater than or equal to a pre-determined threshold. In an embodiment, the threshold is 0.9.

In S809, if the similarity of the target node with one of its neighbour nodes is above the preselected threshold (e.g. 0.9), then the direct hyponyms and direct hypernyms of this neighbour node are found, and their similarity with the target node is computed and compared to the threshold. This process is repeated.

Thus in S809, as long as the similarity of a node with the initial concept (target node) is above the threshold, the process continues walking the graph and computing the similarity with the initial node. Alternatively, the process may be continued until a maximum number of similar nodes is reached, for example 60 nodes. In FIG. 8(b), a first stage of walking the graph is represented in the lightest shading, the second level in the darker shading. The similarity values to the concept to be embedded, for each of the first degree neighbours and second degree neighbours, are shown. If the node similarity with the initial concept is above 0.9, the process continues exploring the graph in that direction by extracting its hypernyms and hyponyms. FIG. 8(b) shows a visualisation of the process of extracting the similar nodes by walking the graph in every direction until a node below the threshold is encountered.

At the end of this process, all the nodes in the graph with a similarity with the initial node above the threshold are extracted. In S810, among these nodes, the existing embeddings of the nodes are extracted. In S811, these embeddings are then used to determine an embedding for the target node. For example, these embeddings may be averaged to compute an embedding of the initial concept (target node). Other methods of determining the embedding for the target node from the embeddings of the similar nodes can be used, for example by determining a weighted average based on distance or similarity.

Using this method, embeddings can be determined for most of the 1,640,384 nodes present in the knowledge base. In practice, some nodes don't have similar neighbours with an existing embedding, so it may not be possible to compute a new embedding for all nodes.

FIG. 8(c) shows an example of the combined similarity scores for part of the knowledge graph. The similarity of each node with the “Acute severe exacerbation of asthma co-occurrent and due to allergic asthma” node (indicated by the arrow) has been computed. The nodes with a star are the ones with an embedding generated using the word embedding model. The other embeddings are computed using the combined method. The scale on the left hand side shows the similarity, with the node shading indicating its value on the scale.

There are multiple hyperparameters of the model which has been described in relation to FIG. 8:
Different algorithm types can be used (e.g. word2vec or Fastext);
Embeddings with different sizes can be used (e.g. 32, 128 or 512);
Different types of similarity measures can be used (Jaccard, Dice, Ochiaï, Simpson, Braun-Blanquet, Sokal and Sneath);
When aggregating the embeddings from the similar nodes in the graph, the similarity threshold to consider a node to be similar can be varied;
The maximum number of similar nodes to be picked (e.g. 60) can be varied, where used.

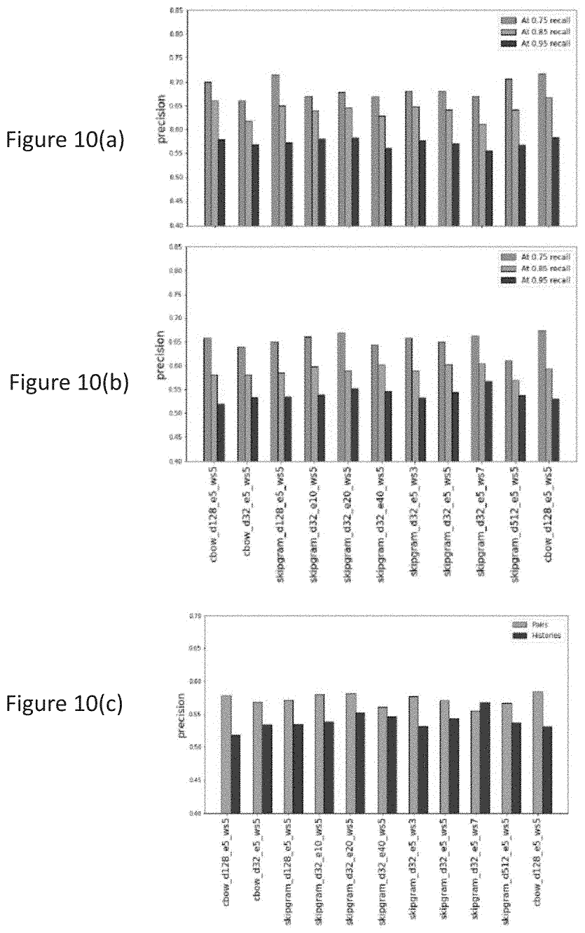

These may be varied in order to improve performance, as is discussed in relation to FIG. 10 below.

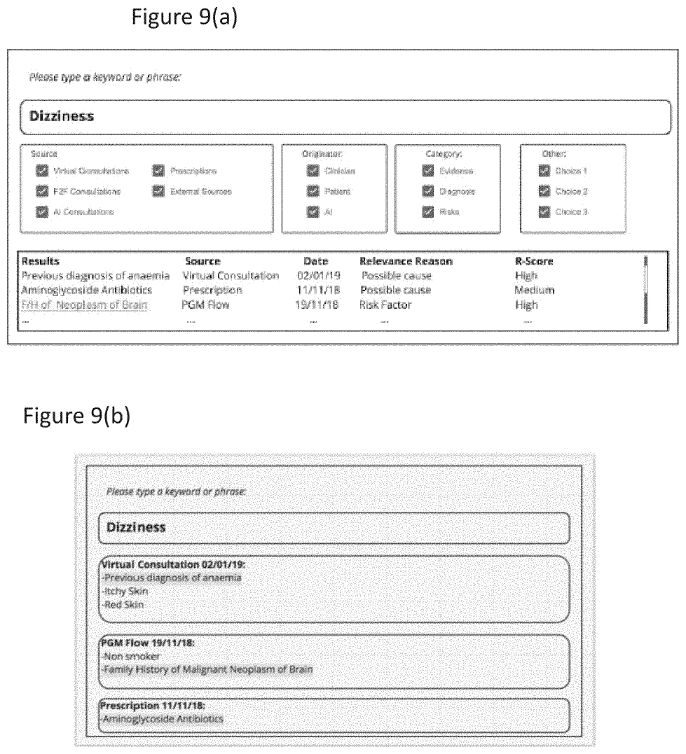

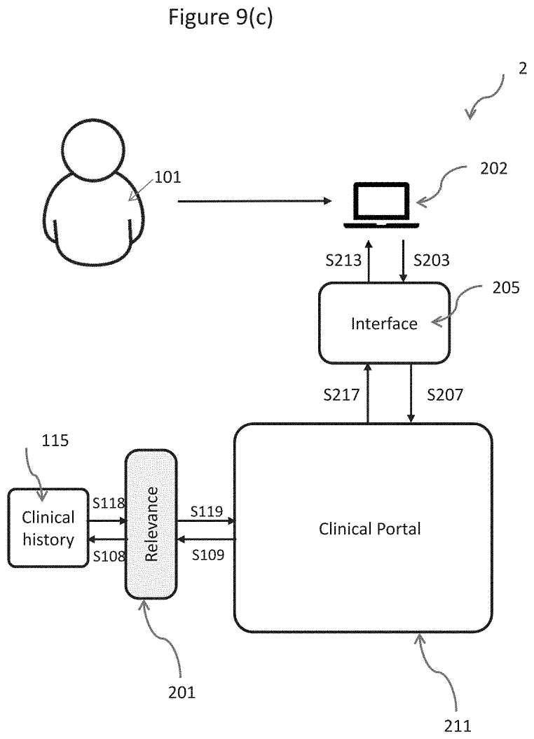

Although in the above, examples of use of the relevance module 201 in a diagnosis system have been described, alternatively, the relevance system 201 can be used separately to a diagnosis system. For example, the relevance module 201 may be used as part of a clinical portal system. A schematic illustration of a clinical portal system 2 in accordance with an embodiment is shown in FIG. 9(c). The system comprises a user interface 205 that talks to a clinical portal backend 211. The clinical portal backend 211 interacts with clinical history 115 through a relevance module 201.

A user 101, for example a GP or other healthcare professional, communicates with the system 2 via a computing device 102, which may be a laptop or fixed computer for example.

The device 202 communicates with interface 205, and transmits the information corresponding to the user input to the interface 205 in S203. Interface 205 has two primary functions, the first function is to receive the information input by the user. In step S207, this information is passed on to the clinical portal 211. The second function is to take the output of the clinical portal 211 and to send this back to the device 202 in steps S217 and S213.

The user 101 inputs information identifying a patient. The user input also comprises at least one input concept, for example a symptom experienced by the patient. In step S203 this is sent to interface 205. This information is then sent to the clinical portal 211 in step S207. The clinical portal 211 extracts a list of concepts which are present in the user input. This may correspond to a list of identifiers. For example, in this step, following receipt of an input phrase, the clinical portal 211 calls an NLP dependency parser which extracts one or more symptoms which are present in the phrase, and outputs the concept(s) corresponding to the symptom(s).

As described previously, a stored set of information relating to items of medical data is stored in the clinical history 115. The clinical portal 211 sends a request to the relevance module 201, together with the information identifying the patient and the user input. The relevance module 201 then obtains the information relating to the patient from the clinical history 115, based on the patient identification. The information stored in the clinical history 115 has been described previously.

The relevance module 201 takes as input the information from the clinical history 115 relating to the patient. The relevance module 201 then determines whether each concept for which information is stored relating to the patient in the clinical history is relevant to the user input. Specifically, for each concept in the user input, the relevance module 201 determines a measure of relevance for each concept extracted from the clinical history 115 for the patient. Examples of how this relevance may be determined have been described above, in relation to FIGS. 2(b) and (c).

The relevance module 201 then passes information relating to the relevant symptoms, risk factors and/or diseases back to the clinical portal 211. The relevance module 201 acts as an interface between the clinical portal 211 and the patient's stored clinical history 115 that pre-processes information for items of medical data stored in the clinical history.

The relevance module 201 may output the set of as representing the relevant symptoms, risk factors and/or diseases, together with the information (for example, the information may indicate whether the concept is present or absent). The clinical portal 211 may then transmit back this information in step S217. The interface 205 can then supply this information back to the device 202 of the user in step S213. The output displayed on the user device 202 comprises the information corresponding to the relevant concepts from the clinical history 115. For example, it may comprise a list of relevant concepts which are indicated as present in the clinical history 115, or a list of relevant concepts which are indicated as present or absent for the patient in the clinical history 115. The relevance feature is configured to extract relevant data from the patient health record stored in the User Graph 115.

In some embodiments, only relevant information is transmitted to the clinical portal 211. Retrieving information from the clinical history 115 may provide additional evidence that was not reported by the patient that allows the user to make a diagnosis. The clinical history is likely to comprise a large data set however. Some of the information derived from the clinical history 115 may not be relevant. If irrelevant information is included, this results in transmission of a large amount of data to the clinical portal 211. It may also result in transmission of a large amount of data to the user device 202. The data may therefore be pre-processed so that information determined to be relevant is transmitted. The system pre-processes the information from the stored information to identify a sub-set of relevant information.

When using a clinical portal, the clinician may scroll through the patient medical history until they find the information that will help them to make an informed decision about the patient diagnosis. This may include deep-diving into specific previous Consultation Notes or Symptom Checker chat histories if necessary. Time may be spent by clinicians searching for specific information they deem to be relevant to their patient's presenting complaint. Furthermore, being presented with so much information at once can overwhelm clinicians, exacerbating the risk of error resulting in omission of key information from decision-making. Including the relevance module 201 feature can help to mitigate both these problems.

In some embodiments, information from the clinical history may be provided ordered by the relevance measure, such that the more relevant information is provided first. The relevance module 201 may therefore act to reduce the total number of requests produced when the user is navigating the patient history. For example, when used together with paginated search, displaying the relevant results reduces the number of pages retrieved on average before the entry of interest is obtained.

The concept embeddings are generated during the training stage and stored in memory, the relevance module 201 may comprise a storage unit in which the concept embeddings are stored. The measure of relevance can therefore be calculated efficiently for each input concept. The concept embeddings are pre-computed, so the relevance module can be scaled horizontally for efficiency and reliability.

A GP may interact with the relevant clinical history at various points during a consultation, for example, where a GP has a diagnosis in mind, and they want to retrieve any relevant evidence/risk factors (to that diagnosis) in the patient clinical history. In this case, concepts relevant to the suspected diagnosis are retrieved (rather than concepts relevant to the patient input), in the same manner as described above. Alternatively, a GP may want to know if a patient has experienced a given symptom (or a related one) in their clinical history. In this case, relevance to a diagnosis concept may be determined.

FIG. 9(a) shows an example of how relevance might appear to a user accessing the Clinical Portal. The user inputs a keyword or phrase, in this case dizziness. The relevance module 201 then obtains the relevant concepts from the clinical history, together with the information relating to these concepts. These are then displayed in the results section. For example, only the relevant concepts may be displayed in the results section, as shown in FIG. 9(a). Alternatively, all of the concepts from the clinical history may be displayed, with the relevant concepts highlighted, as shown in FIG. 9(b). The relevance module 201 provides the capability to filter for or highlight the most relevant concepts given a presenting scenario (e.g. a patient presenting symptom, or a suspected diagnosis).

A Clinical Portal 211 may provide information from the user history 115 to be displayed in a chronological manner, with data coming from a variety of sources. The User Graph, or clinical history 115, collects data from a variety of sources and stores it. The User Graph Timeline API provides this data in a chronological order. The Clinical Portal 211 is integrated with the User Graph Timeline, or clinical history 115. In an embodiment, it extracts only diagnosis and Check Base flows from the clinical history 115, and displays them to the GP. Other data from the patient health record is extracted directly from the corresponding services (e.g. prescriptions data is extracted from the Prescription Service).

In an embodiment, the relevance module 201 aims to capture how much information a recommendation can provide to the doctor and rank those entries the highest.

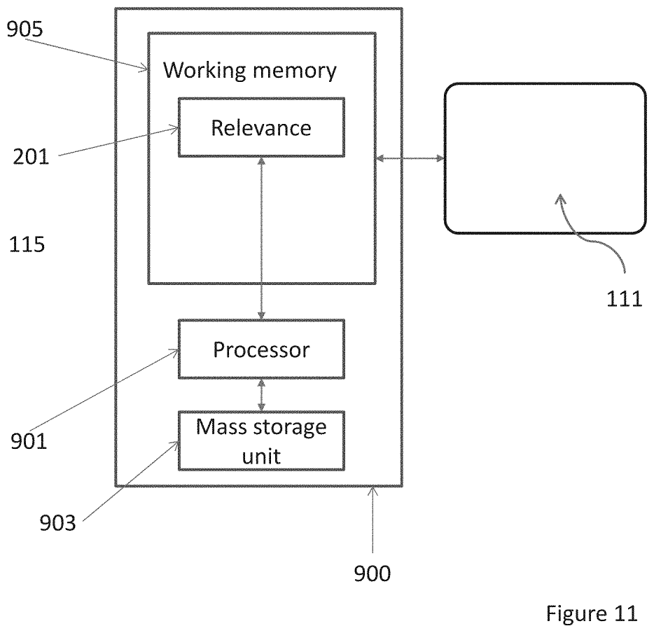

While it will be appreciated that the above embodiments are applicable to any computing system, an example computing system is illustrated in FIG. 11, which provides means capable of putting an embodiment, as described herein, into effect. As illustrated, the computing system 900 comprises a processor 901 coupled to a mass storage unit 903 and accessing a working memory 905. As illustrated, a relevance module 201 is represented as a software product stored in working memory 905. Further functionality, such as the validity module 301 may also be embodied as a software product stored in working memory 905. The clinical history may be stored in a database table in the mass storage unit 903 for example, or in external memory, for example in an external computing system.

It will be appreciated that elements of the relevance module 201 may, for convenience, be stored in the mass storage unit 903. It will also be appreciated that the computing system 900 may be connected to and configured to communicate with other parts of a medical diagnosis system 1, such as the diagnosis engine 111, or with other parts of a clinical portal system 2, such as the clinical portal 211. It will also be appreciated that the diagnosis engine 111 may be put into effect by means of a computing system similar to the system 900, or may be put into effect by means of the computing system 900 itself. Furthermore, the clinical portal 211 may be put into effect by means of a computing system similar to the system 900, or may be put into effect by means of the computing system 900 itself.

In use, the system receives data from a user. The programs, including the relevance module, are then executed on the processor in the manner which is described with reference to the above figures. The processor may comprise logic circuitry that responds to and processes the program instructions.

Testing of concept embeddings generated using an example method as described in relation to FIGS. 7 and 8 was performed using a first dataset and a second dataset. The first dataset comprised a set of manually annotated concept pairs and the second dataset comprised a set of manually annotated consultation histories. Both were built from data in the training dataset, and annotated by doctors.

The first dataset of annotated pairs built for testing comprised a list of 4000 pairs of concepts with their respective relevance coefficient added by doctors. Generating the first dataset comprised building pairs and then asking doctors to rate their relevance.

Specifically, generating the first dataset comprised:
1. Extracting symptoms and diagnosis data grouped by users from the EHR database, i.e. acquiring user medical histories;
2. Sampling concept pairs from this data, e.g. by using co-occurrence as described below;
3. Input these pairs in a table and send them for annotation to doctors;
4. Doctors rate the relevance on a scale from 0 to 3;
5. Collect and verify the distribution of the annotated pairs, where score from 0-1 are considered irrelevant and scores from 2-3 are considered relevant.

The method adopted to extract the pairs was to use co-occurrence matrices computed on the training dataset. The co-occurrence matrix represents the number of times one concept in the rows is reported for the same patient as one concept in the columns, in the EHR database. These matrices were stratified by age, sex and computed for different time windows. By sampling the pairs using the co-occurrence matrices instead of randomly taking pairs of non-relevant concepts, the dataset is skewed toward a more balanced distribution of the relevance score. The co-occurrence matrix for conditions occurring within a time window of one day was used. This matrix was extracted as a list of pairs, with their respective frequency of occurrence. This list was sorted based on the frequency. A total of 1,345,193 pairs and 34794 concepts was generated. Extracting the top N pairs gives a good coverage of the most frequent pairs, and limiting the number of repetitions of a concept to a given number gives coverage of concepts. For example, a maximum of repetition of 6 represents 9.3% of the pairs and 8.1% of the concepts. 4000 pairs of medical concepts were extracted in this manner.

Five doctors annotated the pairs on a scale from 0 to 3 using these criteria:
0: Completely unrelated concepts: the concepts have nothing in common and no relationship link them. The concept does not add any value to the presenting complaint.
1: There is a correlation between the two concepts, but an established link might not necessarily exist. This may provide better contextual information to the presenting complaint.
2: The 2 concepts are strongly related clinically so that one may lead to the other, or the 2 are concepts that have an established link (e.g. nausea leads to vomiting/Obesity and IHD). This would usually form part of a doctors decision making process.
3: Extremely related concept: the two concepts always occur together clinically, or one cannot happen without the other (e.g. alcoholic liver disease and liver cirrhosis). This is a vital part of the history based on the presenting complaint.

Each of the 4000 pairs was annotated by 3 doctors. These results were aggregated by taking the rounded mean of the 3 annotations. The pairs were then labelled with 0 to indicate not relevant (corresponding to an annotation of 0 to 1) and 1 to indicate relevant (corresponding to an annotation of 2 to 3).

This first dataset is thus extracted from the pairs that are the most represented in the EHR database. However, testing a set of random pairs it is not very close to the actual task, where the pairs are not extracted randomly but from a patient's clinical history.

The second testing dataset was not biased toward very common pairs. To build this data set, a presenting complaint and a patient history from the EHR database was presented to doctors for scoring. A set of around 100 “consultations”, where each consultation has a set of 20-30 pairs, comprising {presenting complaint, concept extracted from patient medical history}, was generated. Specifically, generating the second dataset comprised:
1. Acquiring user medical history from an EHR database i.e. extract all the concepts grouped by users from the database;
2. Filter the users with a large enough number of concepts reported (20-30 concepts);
3. Extract a presenting complaint from this same patient history;
4. For each set {input query; patient history}, ask the doctors to label all of the concepts in the patient history that are related to the input query with 1.

Table 2 below is an example of how this second dataset is constructed. The concepts in the medical histories are majority non-relevant, which is what would be expected from a non-biased dataset. The presenting complaint in this case is “lumbar pain”.

TABLE 2
Example data used for the second dataset

| Eventdate | Label | In | Is relevant? | Age | Sex |
|---|---|---|---|---|---|
| 2015-08-03 | Pneumonia | | | 63 | F |
| 2015-04-27 | Chest pain | https://bbl.health | [symbol] | | |
| 2015-30-30 | Cough | https://bbl.health | [symbol] | | |
| 2015-01-25 | Fever | https://bbl.health | [symbol] | | |
| 2014-03-13 | Synoope | https://bbl.health | [symbol] | | |
| | symptom | | | | |
| 2013-07-11 | Intertrigo | https://bbl.health | [symbol] | | |
| 2009-07-21 | Nosebleed | https://bbl.health | [symbol] | | |
| 2008-06-29 | Wheezing | https://bbl.health | [symbol] | | |
| | symptom | | | | |
| 2005-01-07 | Lipoma | https://bbl.health | [symbol] | | |
| 2004-06-15 | Prenatal care | https://bbl.health | [symbol] | | |
| 2004-01-08 | Pruritus ani | https://bbl.health | [symbol] | | |
| 2003-07-11 | invert nipple | https://bbl.health | [symbol] | | |
| 2003-03-21 | Carpal canal | https://bbl.health | [symbol] | | |
| 2003-01-06 | Backache | https://bbl.health | [symbol] | | |
| 1995-11-03 | Cholangitis | https://bbl.health | [symbol] | | |
| 1991-01-31 | Prostatism | https://bbl.health | [symbol] | | |

The second data-set was converted into pairs, each pair comprising the presenting complaint and a concept from the patient history, and each pair being labelled as 1 if relevant and 0 if not relevant.

After separating the pairs from the dataset into relevant and non-relevant, the datasets were balanced. The second testing set showed a much higher proportion of non-relevant conditions, where 87% of the conditions in the medical histories were not relevant to the presenting complaint. As a result, a model that returns that all of the conditions as non-relevant will be right 87% of the time. To correct that bias, the dataset is balanced by duplicating pairs of the non-predominant category, i.e. relevant in this case.

The following metrics were used to assess the embeddings:
Precision: fraction of relevant instances among the retrieved instances:

$$Precision = \frac{TP}{TP + FP}$$

where TP is the number of True Positive classifications (where the pair is annotated as relevant and the cosine similarity returns a result classed as relevant) and FP the number of false positive classification (where the pair is annotated as irrelevant and the cosine similarity returns a result classed as relevant).
Recall: fraction of relevant instances that have been retrieved over the total amount of relevant instances:

$$Recall = \frac{TP}{TP + FN}$$

where FN is the number of False Negative classification (where the pair is annotated as relevant and the cosine similarity returns a result classed as irrelevant).
F1 score: the harmonic mean between precision and recall. The range for F1 score is [0,1]. It tells you how precise the classifier (i.e. the result of the cosine similarity combined with the threshold for determining whether this result indicates relevance) is (how many instances it classifies correctly), as well as how robust it is (it does not miss a significant number of instances):

$$F 1 = 2 \cdot \frac{1}{\frac{1}{precision} + \frac{1}{recall}}$$

Mean absolute error: average of the difference between the real value and the predicted value:

$$MAE = \frac{1}{n} \cdot {\sum }_{i = 1}^{n} \vert {y}_{i} - {\hat{y}}_{i} \vert$$

where n is the number of predicted values (number of pairs), yᵢ the i-th true value (annotation, i.e. 1 or 0) and ŷᵢ its corresponding predicted value (i.e. the output from the model).
Mean square error: the average of the square of the difference between the real value and the predicted value.

$$MSE = \frac{1}{n} \cdot {\sum }_{i = 1}^{n} {( {y}_{i} - {\hat{y}}_{i} )}^{2}$$

FIG. 10(a) shows the precision of the word embedding based model described in relation to FIG. 7 for recall values of 0.75, 0.85 and 0.95 tested on the first dataset for different hyperparameters. The recall is set by altering the threshold at which the cosine similarity is determined to be relevant. The different hyperparameters include the type of model (cbow or skip-gram), the dimension of the embeddings (d), the epoch number (e) and the window size (ws).

FIG. 10(b) shows the precision of the word embedding based model for recall of 0.75, 0.85 and 0.95, tested on the second dataset for the different hyperparameters. FIG. 10(c) shows the precision of the different word embedding based models for recall of 0.95 tested on the first dataset and second dataset.

The results of the relevant classification on each of the models tested on the two annotated datasets are shown below. Precision is given for a recall of 75, 85 and 95% with the corresponding classification threshold t used to classify the result of the cosine similarity measure as relevant or not relevant. The F1 score is also given for recall of 75, 85 and 95%. Table 3 shows the results for an example model based on the method described in relation to FIG. 7, and Table 4 shows the results for an example model based on the method described in relation to FIG. 8.

TABLE 3
testing results for example model such as described in relation to FIG. 7

| Word | Precision: 70.30%, recall: | Precision: 62.46%, recall: |
|---|---|---|
| embedding | 75.04% | 74.94% |
| model | (t = 0.495) | (t = 0.146) |
| (word2vec, 128 | Precision: 66.52%, recall: | Precision: 56.02%, recall: |
| dimensions) | 84.85% | 84.85% |
| | (t = 0.414) | (t = 0.086) |
| | Precision: 59.95%, recall: | Precision: 51.03%, recall: |
| | 95.07% | 95.16% |
| | (t = 0.278) | (t = 0.023) |
| | Mean Absolute Error: 0.26 | Mean Absolute Error: 0.40 |
| | Mean Square Error: 0.10 | Mean Square Error: 0.27 |
| | F1 score at recall 75%: | F1 score at recall 75%: |
| | 0.725 | 0.661 |
| | F1 score at recall 85%: | F1 score at recall 85%: |
| | 0.745 | 0.674 |
| | F1 score at recall 95%: | F1 score at recall 95%: |
| | 0.735 | 0.664 |

TABLE 4
testing results for example model such as described in relation to FIG. 8

| Combined | Precision: 74.69%, recall: | Precision: 66.14%, recall: |
|---|---|---|
| model | 75.04% | 74.93% |
| (word | (t = 0.543) | (t = 0.124) |
| embedding | Precision: 68.66%, recall: | Precision: 56.76%, recall: |
| and | 85.19% | 84.94% |
| knowledge- | (t = 0.411) | (t = 0.065) |
| base | Precision: 61.41%, recall: | Precision: 51.14%, recall: |
| similarity) | 95.11% | 94.73% |
| | (t = 0.026) | (t = 0.18000000000000002) |
| | Mean Absolute Error: 0.25 | Mean Absolute Error: 0.38 |
| | Mean Square Error: 0.10 | Mean Square Error: 0.26 |
| | F1 score at recall 75%: | F1 score at recall 75%: |
| | 0.746 | 0.702 |
| | F1 score at recall 85%: | F1 score at recall 85%: |
| | 0.760 | 0.680 |
| | F1 score at recall 95%: | F1 score at recall 95%: |
| | 0.746 | 0.664 |

While certain embodiments have been described, these embodiments have been presented by way of example only, and are not intended to limit the scope of the inventions. Indeed the novel methods and apparatus described herein may be embodied in a variety of other forms; furthermore, various omissions, substitutions and changes in the form of methods and apparatus described herein may be made.

1. A computer-implemented method for medical diagnosis, comprising:
receiving a user input from a user, the user input comprising an input symptom;
determining a measure of relevance of a plurality of items of medical data to the user input, wherein the plurality of items of medical data are items of medical data for which information associated with the user is stored;
determining whether to include the stored information corresponding to an item of medical data in a first set of information, based on the measure of relevance for the item of medical data;
providing the user input and the first set of information as an input to a model, the model being configured to output a probability of the user having a disease; and
outputting a diagnosis based on the probability of the user having a disease.

2. The method according to claim 1, wherein determining the measure of the relevance of an item of medical data to the user input comprises:
obtaining a first vector representation corresponding to the input symptom;
obtaining a second vector representation corresponding to the item of medical data;
determining a similarity measure between the first vector representation and the second vector representation.

3. The method according to claim 2, wherein the similarity measure is the cosine similarity.

4. The method according to claim 1, wherein the input is received from a user device, the method further comprising:
sending the first set of information to the user device;
receiving confirmation information corresponding to the first set of information from the user device.

5. The method according to claim 2, wherein when the user input comprises two or more input symptoms, determining the measure of the relevance of an item of medical data to the user input comprises:
obtaining a first vector representation corresponding to each of the input symptoms;
obtaining a second vector representation corresponding to the item of medical data;
determining a similarity measure between each first vector representation and the second vector representation;
determining an average similarity measure.

6. The method according to claim 1, wherein determining whether to include the information corresponding to an item of medical data in a first set of information based on the measure of relevance for the item of medical data comprises determining whether the measure of relevance meets a pre-determined threshold.

7. The method according to claim 1, wherein determining whether to include the information corresponding to an item of medical data in a first set of information based on the measure of relevance for the item of medical data comprises determining whether the measure of relevance is within a pre-determined number of the most relevant.

8. A method according to claim 1, wherein an item of medical data comprises a symptom, risk factor, disease, physiological data, recommendation or behaviour.

9. A method according to claim 1, wherein said model comprises a probabilistic graphical model comprising probability distributions and relationships between symptoms and diseases, and an inference engine configured to perform Bayesian inference on said probabilistic graphical model, and wherein determining the probability that the user has a disease comprises performing approximate inference on the probabilistic graphical model to obtain a prediction of the probability that the user has a disease.

10. The method according to claim 9, further comprising:
obtaining a set of items of medical data to be used in the probabilistic graphical model;
obtaining stored information associated with the user relating to the items of medical data to be used in the model;
determining the measure of the relevance for the items of medical data.

11. A method according to claim 10, wherein inference is performed using a discriminative model, wherein the discriminative model has been pre-trained to approximate the probabilistic graphical model, the discriminative model being trained using samples generated from said probabilistic graphical model, wherein some of the data of the samples has been masked to allow the discriminative model to produce data which is robust to the user providing incomplete information about their symptoms,
and wherein determining the probability that the user has a disease comprises deriving estimates of the probabilities that the user has that disease from the discriminative model, inputting these estimates to the inference engine and performing approximate inference on the probabilistic graphical model to obtain a prediction of the probability that the user has that disease.

12. The method according to claim 1, further comprising:
determining the validity of the stored information;
wherein determining the measure of the relevance of the plurality of items of medical data comprises determining the measure of relevance for the items of medical data for which the stored information is valid.

13. A method according to claim 12, wherein the validity is determined from information indicating which items of medical data are permanently valid.

14. A medical diagnosis system comprising:
a user interface configured to receive a user input from a user, the user input comprising at least one input symptom;
one or more processors configured to:
determine a measure of relevance of a plurality of items of medical data to the user input, wherein the plurality of items of medical data are items of medical data for which information associated with the user is stored;
determine whether to include the information corresponding to an item of medical data in a first set of information, based on the measure of relevance for the item of medical data;
provide the user input and the first set of information as an input to a model, the model being configured to output a probability of the user having a disease; and
a display device, configured to display a diagnosis based on the probability of the user having a disease.

15. A computer implemented method of training a medical diagnosis system, comprising:
obtaining a dataset comprising a plurality of items of medical data associated with each of a plurality of patients;
learning a vector representation corresponding to items of medical data in the dataset;
storing the vector representation associated with the item of medical data.

16. The method according to claim 15, wherein the vector representations are learned by training a model to reconstruct the context of concepts from the dataset.

17. The method according to claim 16, further comprising:
obtaining an ontology comprising items of medical data and information describing the relationships between the items of medical data;
for a target item of medical data, determining one or more items of medical data in the ontology which are similar to the target item and for which there is an associated stored vector representation;
determining a vector representation for the target item of medical data from the vector representations of the one or more similar items of medical data.

18. The method according to claim 17, wherein an item of medical data is determined to be similar to a target item using an information content based similarity measure.

19. A non-transitory carrier medium comprising computer readable code configured to cause a computer to perform the method of claim 1.

20. A non-transitory carrier medium comprising computer readable code configured to cause a computer to perform the method of claim 15.
